(19) United States
(12) Patent Application Publication
(10) Pub. No.: US 2016/0067195 A1
(43) Pub. Date: Mar. 10, 2016

(54) METHOD FOR TESTING RISK OF MULTIPLE SYSTEM ATROPHY, TEST KIT, AND DRUG FOR THE TREATMENT OR PREVENTION OF MULTIPLE SYSTEM ATROPHY

(71) Applicant: The University of Tokyo, Tokyo (JP)

(72) Inventors: Shoji TSUJI, Tokyo (JP); Jun MITSUI, Tokyo (JP)

(21) Appl. No.: 14/763,794

(22) PCT Filed: Feb. 5, 2014
(86) PCT No.: PCT/JP2014/052658

§ 371 Date: Nov. 13, 2015

(30) Foreign Application Priority Data
Feb. 5, 2013 (JP) 2013-020763

(51) Int. Cl. A61K 31/122; G01N 33/68; C12Q 1/68

(52) U.S. Cl. CPC A61K 31/122; C12Q 1/6883; G01N 33/6896; G01N 2800/2814; C12Q 2600/158

(57) ABSTRACT

An object of the present invention is to elucidate the onset mechanism of MSA through specification of a causative gene of it and further, to find a treatment method of it. The present invention provides a method for testing a multiple system atrophy risk of a test subject including a step of detecting a variant that deteriorates the biosynthesis of coenzyme Q10 in a sample collected from the test subject. Examples of the variant that deteriorates the biosynthesis of coenzyme Q10 include variants that suppress the expression or function of coenzyme Q2.

### TECHNICAL FIELD

The present invention relates to a method for testing the onset risk of multiple system atrophy, and the like.

### BACKGROUND ART

Multiple system atrophy (MSA) is one of intractable neurodegenerative diseases. The average onset age of it is 57.5±7.2 and clinical symptoms of it appear as various combinations of cerebellar ataxia, parkinsonism, autonomic symptoms, and pyramidal tract signs. It is classified into two clinical disease subtypes, that is, MSA-C having cerebellar ataxia as a main symptom and MSA-P having parkinsonism as a main symptom. MSA-C corresponds to olivopontocerebellar atrophy, while MSA-P corresponds to striato-nigral degeneration in previous nomenclature. In addition, there is a Shy-Drager symptom group having autonomic failure as a main symptom. In the Japanese population, MSA-C is more frequently observed than MSA-P, while in Westerners MSA-P is more frequent.

Pathologically, it is characterized by glial cytoplasmic inclusion (GCI), where α-synuclein accumulate as a main component.

MSA is considered to be a typical sporadic neurodegenerative disease without any familial occurrence. Occurrence of multiplex families with MSA has however been found, though they are very rare (Non-patent Documents 1 to 3), suggesting participation of genetic factors in it.

The onset mechanism of MSA remains unknown and only symptomatic treatment has been performed.

### CITATION LIST

### Non-Patent Documents

Non-patent Document 1: Hara K, et al. Arch Neurol 2007; 64:545-51.
Non-patent Document 2: Wullner U, et al. J Neurol Neurosurg Psychiatry 2009; 80:449-50.
Non-patent Document 3: Hohler AD and Singh VJ. J Clin Neurosci 2012; 19:479-80.

### DISCLOSURE OF THE INVENTION

### Problem to be Solved by the Invention

An object of the present invention is to elucidate the onset mechanism of MSA by identifying a causative gene of it and find the treatment method of it.

### Means for Solving the Problem

The present inventors have proceeded with a study in order to achieve the above-mentioned object. As a result, it has been found that multiplex families with MSA have a carrier of variants of para-hydroxybenzoate-polyprenyltransferase gene (para-hydroxybenzoate-polyprenyltransferase (EC 2.5.1.39) may hereinafter be called “coenzyme Q2” or “CoQ2” and its gene may be called “COQ2” or “COQ2 gene”) and has been confirmed that, if homozygous or compound heterozygous COQ2 variants exist, some of familial multiple-system atrophy patients develop this disease with the variant as a sufficient condition. It has also been elucidated that also in sporadic MSA, a heterozygous COQ2 variant becomes a large risk factor for MSA onset. Further, since COQ2 takes part in biosynthesis of CoQ10, the amount of CoQ10 in the tissue of familial MSA patients was measured to prove its reduction. It has therefore been confirmed that the above result logically and strongly suggests the therapeutic effect of CoQ10 administration, leading to the completion of the invention.

The present invention therefore relates to:

[1] a method for testing the risk of multiple system atrophy of a test subject, including a step of detecting a variant that deteriorates biosynthesis of coenzyme Q10 in a sample collected from the test subject;

[2] the method as described above in [1], wherein the variant that deteriorates biosynthesis of coenzyme Q10 is a variant that suppresses expression or function of para-hydroxybenzoate-polyprenyltransferase (coenzyme Q2);

[3] the method as described above in [2], wherein the variant that suppresses expression or function of coenzyme Q2 is selected from the group consisting of P49H, S57T, R69H, M78V, I97T, P107S, S113F, T267A, S297C, R337Q, R337X, and V343A in SEQ ID NO: 1;

[4] the method as described above in [2], wherein the variant that suppresses expression or function of coenzyme Q2 is V343A;

[5] a test kit of multiple system atrophy, including at least one of the followings (i) to (iii):

(i) a nucleic acid that hybridizes with a region, in a coenzyme Q2 gene, containing a nucleic acid encoding an amino acid at a site selected from the group consisting of position 49, position 57, position 69, position 78, position 97, position 107, position 113, position 267, position 297, position 337, and position 343 of a coenzyme Q2 protein (SEQ ID NO: 1);

(ii) a primer set capable of amplifying a region, in the coenzyme Q2 gene, containing a nucleic acid encoding an amino acid at a site selected from the group consisting of position 49, position 57, position 69, position 78, position 97, position 107, position 113, position 267, position 297, position 337, and position 343 of a coenzyme Q2 protein (SEQ ID NO: 1); and

(iii) an antibody that binds, without cross-reactivity, only to either one of a coenzyme Q2 protein having at least one variant selected from the group consisting of P49H, S57T, R69H, M78V, I97T, P107S, S113F, T267A, S297C, R337Q, R337X, and V343A in SEQ ID NO: 1 or a wild type coenzyme Q2 protein;

[6] a drug for the prevention or treatment of multiple system atrophy containing coenzyme Q10;

[7] a method of preventing or treating multiple system atrophy, including a step of administering coenzyme Q10;

[8] a method of screening a drug for the prevention or treatment of multiple system atrophy, including:

a step of contacting each of candidate compounds with a cell and then incubating, and

a step of selecting a candidate compound that increases the amount of coenzyme Q10 in the cell; and

[9] a nucleic acid encoding a coenzyme Q2 protein containing a V343A variant.

### Effect of the Invention

In the present invention, risk of developing MSA can be assessed by an easy method of detecting presence or absence of a predetermined variant in the COQ2 gene of a test subject.

When the risk of developing MSA is high, replenishment with CoQ10 is expected to suppress the onset and in addition, it is strongly suggested that replenishment with CoQ10 is also effective for the treatment of MSA.

### BRIEF DESCRIPTION OF THE DRAWINGS

FIG. 1A shows pedigrees of six multiplex families with MSA. Parents of FMSA_{—}1 were consanguineous (first degree cousins). Both two FMSA_{—}1 patients (II-4 and II-8) suffered from retinitis pigmentosa, but the other brothers suffered from neither MSA nor retinitis pigmentosa. The definite diagnosis of II-4 and II-8 of FMSA_{—}1 and II-6 of FMSA_{—}8 with MSA was carried out through biopsy. Two other brothers of FMSA_{—}8 were PD patients. The □ represents a male member, ∘ represents a female member, a black solid represents an MSA patient, a gray one represents a PD patient, and a blank one represents an unaffected family member. A black dot is a member from which a genomic DNA can be obtained. MSA-C means MSA having cerebellar ataxia as a main symptom; MSA-P is MSA having parkinsonism as a main symptom; and PD means Parkinson disease.

FIG. 1B shows multipoint parametric linkage analysis. Using pipeline software SNPHiTLink, SNPs with a p value >0.05 in Hardy-Weinberg test, a call rate >0.95, a confidence score of genotyping <0.1, a minor allele frequency in the controls >0, and an inter-marker distance from 80 kb to 120 kb for linkage analysis were selected for the linkage analysis. Multipoint parametric linkage analysis (autosomal recessive inheritance with complete penetrance) and haplotype reconstruction were performed using Allegro version 2. Maximum LOD score was 1.93 and a region including a region on Chromosome 4 (from 72.795 Mb to 89.616 Mb at of NCBI36/hg18 assembly), a region on Chromosome 5 (from 149.50 Mb to 168.32 Mb), a region on Chromosome 6 (from 85.499 Mb to 87.382 Mb), a region on Chromosome 7 (from 62.754 Mb to 64.907 Mb), a region on Chromosome 9 (from 99.781 Mb to 115.484 Mb), and a region on Chromosome 13 (from 75.849 Mb to 98.253 Mb) totaled about 80 Mb.

FIG. 1C is an explanatory view of a procedure of narrowing down candidate variants. By whole genome sequencing, 3,492,929 in total of SNVs and indels were found and 54,306 of them were located in the candidate regions. Of these, 342 regions encoded an exon or splice site, 78 regions of which were nonsynonymous or splice site variants. Of these, only four SNVs were not registered in dbSNP130 and therefore novel.

FIG. 1D shows the results of direct sequence of FMSA_{—}1 patient (II-4, upper panel) and non-affected patient (II-7, lower panel). The patient had homozygous M78V-V343A.

FIG. 2A shows the results of yeast complementation assay of COQ2 variants. The left panel shows growth curves of a yeast coq2 null variant transformed with a pAUR123 vector containing wild type (wt) human COQ2 gene or a mock vector. In the center panel, shown are growth curves of yeast coq2 null variants transformed with pAUR123 vectors containing a human COQ2 cDNA having various variants (L16V, P22L, F29L, N336H, V343A, I97T, T267A, or S297C). Compared with the wild type COQ2, the variants I97T, T267A, and S297C have a greatly decreased growth rate, but exhibited a higher growth rate than the coq2 null strain (moderately deleterious variants). L16V, P22L, F29L, N336H, and V343A showed a growth rate nearly equal to that of the wild type COQ2. The right panel shows growth curves of a coq2 null variant transformed with pAUR123 vectors containing a human COQ2 cDNA having various variants (P49H, S57T, R69H, M78V, M78V-V343A, P107S, S113F, R337X, or R337Q). Similar to the coq2 null strain, they showed a marked reduction in respiration dependent growth rate (severely deleterious variants). Each yeast strain was pre-cultured in a YPD medium, diluted to give an OD600 of 0.1, and incubated in a yeast extract•peptone•glycerol (YPG) medium at 23° C. for 4 days with shaking at a rate of 200 times/minute. The OD600 was measured every 10 minutes and plotted based on incubation time. The CoQ2 activity was determined by measuring the incorporation of radioactive parahydroxybenzoate (PHB) in decaprenyl PHB.

FIG. 2B shows the measurement results of enzyme activity of COQ2 variants. The CoQ2 activity in lymphoblastoid cells obtained from MSA patients carrying any of variants of the COQ2 gene (R337Q/V343A, R337X/V343A, V343A/V343A, or V343A/wt) and control subjects having no variant was measured. The enzyme activity (pmol/mg-protein/minute) of each subject is indicated by the central value (column) and the standard deviation (bar) of the test made nine times independently. Group comparison was performed using the Kruskal-Wallis test, followed by the Steel test for multiple testing. Asterisks indicate p<0.05 for comparison with one of the controls (wild type COQ2 genotype).

FIG. 2C shows the measurement results of the CoQ10 concentrations in frozen cerebellum samples of MSA patients. The CoQ10 concentrations in frozen cerebellum samples obtained from three MSA patients (one carrying M78V-V343A/M78V-V343A and two carrying V343A/wt) and three controls (wt/wt). About 100 mg of the brain tissue was homogenized in 10 volumes (volume to weight) of 10 mM Tris HCl (pH 7.4) containing 0.32M sucrose, 1 mM EDTA, and 20% SDS. Then, CoQ10 was extracted in hexane/ethanol (5:2 v/v). The CoQ10 concentration in the extract was measured using HPLC, followed by regulation with a free cholesterol concentration.

FIG. 3 is FIG. 1 of Andrew J. et al., The American Journal of Human Genetics 84, 558-566, May 15, 2009. It shows the outline of the biosynthesis of CoQ10.

FIG. 4 shows the outline of a clinical trial of ubiquinol made for MSA patients.

FIG. 5 shows the measurement results of the plasma CoQ10 concentration (μg/ml) after administration of each dose of ubiquinol.

FIG. 6 shows the measurement results of total CoQ10/free cholesterol (nM/μM) in mononuclear cells after administration of each dose of ubiquinol.

FIG. 7 shows measurement results of the CoQ10 concentration (μg/ml) of the spinal fluid after administration of each dose of ubiquinol.

FIG. 8 shows the measurement results of 8-OHdG (ng/mg-Cre) in urine after administration of each dose of ubiquinol.

FIG. 9 shows the clinical evaluation scale of ubiquinol.

FIG. 10 shows the measurement results of cerebral blood flow rate and metabolic rate of oxygen before and after ubiquinol administration.

### DESCRIPTION OF EMBODIMENTS

### Method for Testing Risk of MSA

The method for testing risk of MSA according to the present invention includes a step of detecting a variant that deteriorates biosynthesis of coenzyme Q10 in a sample collected from a test subject.

The term “method for testing risk of MSA” as used herein means a testing method performed to collect data necessary for determining the possibility that the test subject has MSA or determining whether the test subject exhibiting MSA-like symptoms suffers from MSA or not. The testing method of the present invention can be performed by test companies or the like.

The clinical disease type of MSA is not particularly limited, but as will be described later, a specific mode of the method of the present invention is suited for specific detection of MSA-C.

The term “CoQ10” as used herein means a benzoquinone derivative called “ubiquinone”. An oxidized form may be called “ubiquinone” and a reduce form may be called “ubiquinol”. The term CoQ10 as used herein means either the oxidized form or the reduced form.

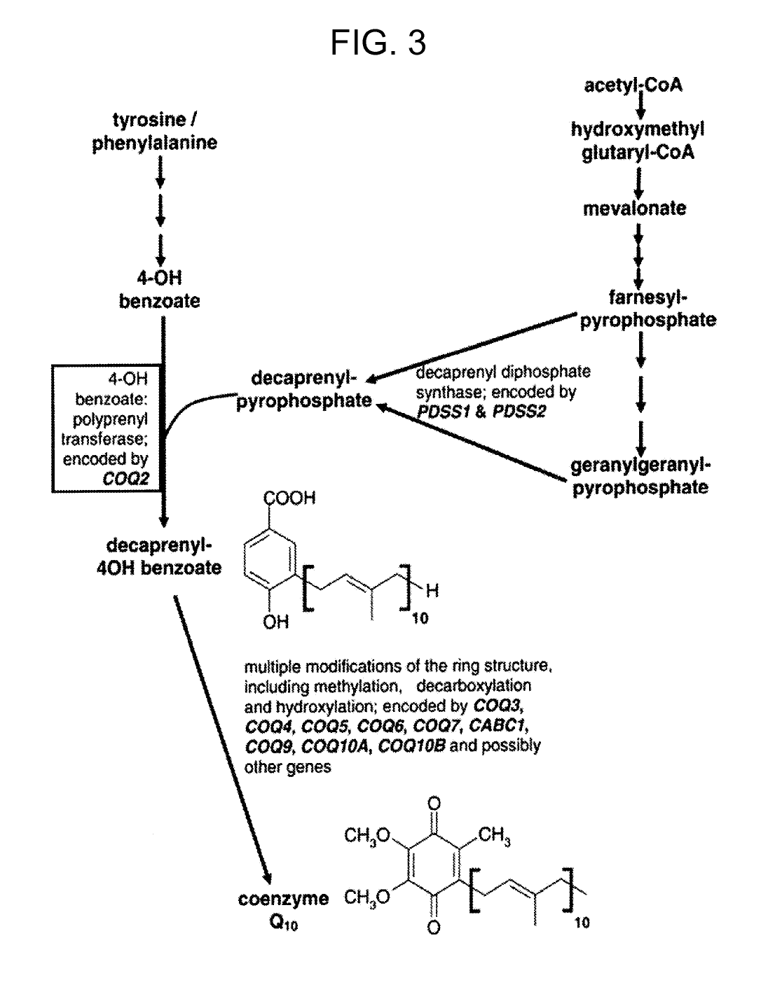

The outline of biosynthetic pathway of CoQ10 is shown in FIG. 3. Prenylation of parahydroxybenzoate (PHB) in the presence of CoQ2 as a catalyst produces decaprenyl PHB. The resulting decaprenyl PHB is subjected to various modifications with many coenzymes to biosynthesize CoQ10.

As will be described later in Examples, the present inventors elucidated that variants in CoQ2 gene that is associated with biosynthesis of CoQ10 take part in MSA risk. It is presumed that not only a variant in CoQ2 but also a variant that deteriorates biosynthesis of CoQ10 has possibility of increasing the MSA risk. Examples of the variant that deteriorates biosynthesis of CoQ10 include, but not limited to, a variant in CoQ2 which will be described later and a variant of various enzymes involved in biosynthesis of CoQ10. Those skilled in the art can select a known variant or newly discovered variant that deteriorates biosynthesis of CoQ10 as needed and detect such a variant.

In this specification, the sample collected from the test subject may be any sample insofar as it allows detection of a variant that deteriorates biosynthesis of CoQ10, examples include blood, other body fluids, skin, tissues, and cells.

An example of the variant that deteriorates biosynthesis of CoQ10 is a variant that suppresses expression or function of CoQ2.

In the specification, the CoQ2 is an enzyme having the following amino acid sequence and is encoded by a CoQ2 gene. It is also called para-hydroxybenzoate-polyprenyltransferase and it catalyzes, in the biosynthesis of CoQ10, a transfer reaction of a decaprenyl group from decaprenyl pyrophosphate to PHB.

(SEQ ID NO: 1)
MLGSRAAGFARGLRALALAWLPGWRGRSFALARAAGAPHGGDLQPPACP
EPRGRQLSLSAAAWDSAPRPLQPYLRLMRLDKPIGTWLLYLPCTWSIGL
AAEPGCFPDWYMLSLFGTGAILMRGAGCTINDMWDQDYDKKVTRTANRP
IAAGDISTFQSFVFLGGQLTLALGVLLCLNYYSIALGAGSLLLVITYPL
MKRISYWPQLALGLTFNWGALLGWSAIKGSCDPSVCLPLYFSGVMWTLI
YDTIYAHQDKRDDVLIGLKSTALRFGENTKPWLSGFSVAMLGALSLVGV
NSGQTAPYYAALGAVGAHLTHQIYTLDIHRPEDCWNKFISNRTLGLIVF
LGIVLGNLWKEKKTDKTKKGIENKIEN

Examples of the variant in CoQ2 that deteriorate biosynthesis of CoQ10 include P49H, S57T, R69H, M78V, I97T, P107S, S113F, T267A, S297C, R337Q, R337X, and V343A. Numbers such as 49 and 57 are the 49^{th} and 57^{th} position of the amino acid sequence represented by SEQ ID NO: 1. The amino acid represented by one letter on the left side of the number is a wild type amino acid residue and the amino acid represented by one letter on the right side of the number is a mutated amino acid residue. In the case where the number has no alphabet on the right side thereof or the number has X as an alphabet on the right side thereof as in R337X, it means that a stop codon is generated in the amino acid portion of the mutated one corresponding thereto and the amino acid sequence thereafter is deleted.

Of these variants, V343A is a variant peculiar to Japanese people. MSA-C is observed more frequently in Japanese people than in Westerners so that it is presumed that the variant V343A has a close relation with MSA-C.

The human COQ2 gene contains, at a first exon thereof, four ATG codons. The amino acid sequence of SEQ ID NO: 1 is obtained from the UniProt database (Q96H96, http://www.uniprot.org/uniprot/Q96H96) in which the fourth codon of these four ATG codons is a translation initiation codon.

The following is a table showing comparison when each variant is annotated using each of NM_{—}015697.7 and NG_{—}015825.1 of NCBI Reference Sequence in which the first ATG codon is a translation initiation codon and BC008804.2 of GenBank.

TABLE 1

| | Amino acid sequence | | mRNA sequence | | Genomic DNA sequence | |
|---|---|---|---|---|---|---|
| | | NCBI Referene | | NCBI Referene | NCBI | Genome |
| | UniProt | Sequence | GenBank | Sequence | Referene | Reference |
| | Q96H96-1 | NM_016697.7 | BC008804.2 | NM_015697.7 | Sequence | Consortium |
| rs ID | (4^{th} ATG codon) | (1^{st} ATG codon) | (4^{th} ATG codon) | (1^{st} ATG codon) | NG_015825.1 | hg19/GRCh37 |
| rs112033303 | 5′ UTR | R22X | N.A. | c.64A > T | g.64A > T | chr4: 84,206,004 |
| rs6818847 | L16V | V66L | c.4GT > G | c.196G > T | g.196G > T | chr4: 84,205,872 |
| N.A. | P22L | P72L | c.65C > T | c.215C > T | g.215C > T | chr4: 84,205,853 |
| N.A. | F29L | F79L | c.85T > C | c.235T > C | g.235T > C | chr4: 84,205,833 |
| N.A. | F49H | P99H | c.146C >A | c.296C > A | g.296C > A | chr4: 84,205,772 |
| N.A. | S57T | S107T | c.170G > C | c.320G > C | g.320G > C | chr4: 84,205,748 |
| N.A. | R69H | R119H | c.206G > A | c.356G > A | g.356G > A | chr4: 84,205,712 |
| N.A. | M78V | M128V | c.232A > G | c.382A > G | g.382A > G | chr4: 84,205,686 |
| N.A. | I97T | I147T | c.290T > C | c.440T > C | g.5837T > C | chr4: 84,200,231 |
| N.A. | P107S | P157S | c.319C > T | c.469C > T | g.5866C > T | chr4: 84,200,202 |
| N.A. | S113F | S163F | c.338C > T | c.488C > T | g.5885C > T | chr4: 84,200,183 |
| rs369627290 | T267A | T317A | c.799A > G | c.949A > G | g.17177A > G | chr4: 84,188,891 |
| N.A. | S297C | S347C | c.889A > T | c.1039A > T | g.17267A > T | chr4: 84,188,801 |
| N.A. | N336H | N386H | c.1006A > C | c.1156A > C | g.20606A > C | chr4: 84,185,462 |
| N.A. | R337X | R387X | c.1009C > T | c.1159C > T | g.20609C > T | chr4: 84,185,459 |
| N.A. | R337Q | R387Q | c.1010G > A | c.1160G > A | g.20610G > A | chr4: 84,185,458 |
| rs148156462 | V343A | V393A | c.1028T > C | c.1178T > C | g.20628T > C | chr4: 84,185,440 |

Variants of these amino acids are presumed to appear based on variants of a nucleic acid so that variants may be detected by detecting variants on a genomic DNA or by detecting variants in RNA or protein. Variants in cDNA may be detected by preparing a cDNA from an RNA derived from a sample of a test subject. Although a method of isolating a DNA or RNA is not particularly limited, a chromosomal DNA or RNA may be extracted and isolated by a method known to those skilled in the art.

In detecting variants in a nucleic acid, those skilled in the art can easily identify, based on the above-mentioned variants of the amino acid, what kind of variants are to be detected.

As a method for detecting variants in a DNA, the following methods can be used.

### (i) PCR-RFLP (Restriction Fragment Length Polymorphism)

This is a method of detecting variants by making use of a difference in the length of a fragment obtained by cleaving a gene having specific variants by a restriction enzyme and this method can be carried out, for example, by the following procedure. A genomic DNA is collected from a test subject and a region containing variants to be detected is amplified by PCR. A restriction enzyme capable of recognizing a mutated site is caused to react with the amplified product and a fragment obtained thereby is isolated and identified by electrophoresis or the like. Presence or absence of cleavage with the restriction enzyme and presence or absence of a variant can be confirmed from the length of the fragment thus obtained. This method may be carried out alternatively by extracting RNA from the sample of a test subject and preparing a cDNA using a reverse transcriptase.

### (ii) Allele-Specific PCR

This is a method making use of the fact that PCR is performed using a primer (allele-specific oligonucleotide: ASO) capable of hybridizing with a variant-including region; and when a sample has a variant, mismatch occurs between the primer and a template DNA and no extension reaction occurs at a high annealing temperature. Presence or absence of a variant in the sample DNA can be found by isolation, identification, and confirmation of the presence or absence of amplification of an amplified product by using electrophoresis or the like.

(iii) Single-Stranded DNA Conformation Polymorphism (SSCP) Method

After amplification by PCR, the resulting DNA is dissociated into a single strand. The single-stranded DNA thus obtained has a specific conformation dependent on a base sequence as a result of various intramolecular interactions including base pairing. Compared with a double-stranded DNA having a stable double helix structure, the conformation of the single-stranded DNA may undergo a change even when there is only one difference in base. Electrophoresis of the single-stranded DNA in polyacrylamide causes a difference in mobility depending on the difference in conformation. The presence or absence of a variant can be determined by detecting the difference in mobility.

More specifically, a chromosomal DNA collected from a test subject is amplified by PCR with a primer labeled with ³²P or the like. The labeled DNA fragment thus obtained is heat-denatured into a single-stranded DNA and the resulting product is isolated using polyacrylamide gel electrophoresis to detect a positional change in band by autoradiography.

### (iv) Denaturing Gradient Gel Electrophoresis (DGGE) Method

This is a method of detecting presence or absence of a variant by making use of easy occurrence of denaturation with a denaturant in the presence of a mismatch in a double-stranded DNA and marked reduction in mobility in electrophoresis when a double-stranded DNA is present in a partially molten form.

More specifically, on a polyacrylamide gel having a concentration gradient of a denaturant such as urea or formaldehyde, a mixture of a DNA sample of a test subject treated with a restriction enzyme or the like if necessary, a normal DNA fragment, and a probe nucleic acid complementary to them is subjected to electrophoresis to separate the sample. In the presence of a mismatch due to a variant, dissociation into a single strand occurs by a denaturant having a lower concentration so that the electrophoresis speed decreases in a gel region having a low denaturant concentration. By comparing with the band of the normal DNA sample, presence or absence of a mismatch can be detected, resulting in detection of the presence or absence of a variant.

The denaturant may have a concentration gradient vertically (vertical gradient method) or in parallel (parallel gradient method). A temperature gradient gel electrophoresis (TGGE) making use of the principle analogous to DGGE has also been developed.

### (v) Method Using a Microarray

This is a method of detecting specific hybridization between a probe DNA immobilized on a microarray and a DNA or RNA sample, and thereby analyzing the presence or absence of a variant. Examples include a method of detecting presence or absence of hybridization and a method of hybridizing the 3′-end of the probe DNA with a site at which a variant is expected to occur, adding a labeled dideoxynucleotide and DNA polymerase, and thereby detecting presence or absence of an extension reaction. The label can be selected from fluorescent dyes, radioactive substances, electrochemically detectable compounds, and the like.

The term “specific hybridization” means specific hybridization under normal hybridization conditions, preferably under highly stringent hybridization conditions. The term “highly stringent conditions” as used herein means, for example, conditions under which at least hybridization is performed, for example, in about 6×SSC/1% SDS solution of 65° C., followed by first washing for 10 minutes in a 20% (v/v) formaldehyde (in 0.1×SSC) of 42° C. and next washing with 0.2×SSC/0.1% SDS of 65° C. The conditions are not limited thereto and those skilled in the art can select the conditions as needed.

### (vi) Pyrosequencing Method

This is a method of detecting a sequencing reaction using chemiluminescence. After the DNA derived from a test subject is amplified by PCR, a single stranded DNA is purified. The resulting DNA and a proper primer are hybridized, followed by addition of deoxynucleotide one base by one base. Pyrophosphoric acid generated by an extension reaction starts a cascade reaction and due to ATP thus generated, light emission of luciferase occurs with luciferin as a substrate. The base sequence of the DNA to be tested can be determined by detecting this light emission and mutation can be detected with high accuracy (Alderborn, A. et al.: Genome Res. (2000) 28:1249-1258).

(vii) Sanger Sequencing

In synthesizing DNAs using a target DNA and a primer, DNAs of various lengths are synthesized by adding any one of four deoxyribonucleotides (dNTP) and one dideoxyribonucleotide (ddNTP) in advance to stop synthesis when the ddNTP is incorporated. The above reaction is made for each of the four dideoxyribonucleotides and DNAs of various lengths are separated from each other by polyacrylamide gel electrophoresis. Since it is possible to identify by polyacrylamide gel electrophoresis even if there is a difference in only one base, the base sequence of the target DNA can be found by detecting the position at which synthesis has stopped. More specifically, each DNA is labeled using various methods and the base sequence is determined through a cycle sequencing reaction using a thermal cycler. Examples of the DNA labeling method include the dye primer method in which the primer is fluorescence-labeled, the dye terminator method in which ddNTP is fluorescence-labeled, and the internal-label method in which a substrate dNTP is labeled.

In the present invention, a target DNA can be obtained by amplifying, by PCR, a region containing a variant to be detected, with a genomic DNA obtained from a test subject as a template.

(viii) Method Using Next-Generation Sequencer

In the present invention, it is also possible to use a method of amplifying, by PCR, a region containing a variant to be detected, with a genomic DNA obtained from a test subject as a template and analyzing the sequence by using a next-generation sequencer. The term “next-generation sequencer” is used in contrast to “first-generation sequencer” using the Sanger method. It is based on various principles, but massively parallel processing of it permits analysis of a large number of base sequences in a short time at a low cost (for example, Holt R. A. and Jones S. J.: Genome Res., Vol. 18 (6):839-864, 2008).

Examples of other methods for detecting a variant include, but not limited to, a method of detecting polymorphism from a difference in mass by using a mass spectrometer (MALDI TOF-MS, etc.); the TaqMan PCR method in which a PCR reaction is performed using a quencher, an allele specific oligo labeled with a fluorescent dye, and a Taq DNA polymerase, followed by typing; a so-called invader method; a rolling circle method; a method of analyzing the sequence of a sample DNA by using a sequencer; the denatured HPLC method; melting temperature analysis; the PCR-SSOP (sequence-specific oligonucleotide probe) method; the PCR-PHFA (preferential homoduplex formation assay) method; and the PCR-RSCA (reference strand conformation assay).

As a method of detecting a variant in the RNA, for example, the following methods can be used.

### (i) Northern Blotting Method

From a sample derived from a test subject, mRNA is taken out by common method and it is heated in a solution containing glyoxal, formamide, formalin, or methyl mercury to eliminate an intramolecular hydrogen bond, destroy the conformation, and form a linear structure. Then, electrophoresis is performed on a formalin-containing agarose gel. The gel is transferred to a nylon membrane or nitrocellulose membrane in a 15 to 20×SSC high salt solution. When a nitrocellulose membrane is used, the RNA is immobilized by the treatment in a vacuum oven at 80° C. for about 2 hours. When a nylon membrane is used, the RNA is immobilized, for example, by exposure to ultraviolet light for crosslinking.

Next, in order to identify a specific mRNA on the membrane, a probe prepared from a cloned cDNA is labeled and the resulting probe and the membrane are brought into contact with each other under specific conditions. Then, an mRNA having the cDNA probe bound thereto can be detected. Hybridization conditions can be selected as needed by those skilled in the art, depending on the salt concentration, temperature, length of the base, composition, or the like.

A trace amount of a target mRNA can be detected by using RT-PCR in combination. Described specifically, a reverse transcription reaction of an RNA sample is performed using a reverse transcriptase and an oligo (dT) primer; the resulting cDNA is amplified by PCR; and a cDNA is detected using a labeled complementary nucleic acid.

### (ii) Dot Blotting

Dot blotting is a modification of Northern blotting and in this method, after modification of an mRNA taken out from a sample derived from a test subject with methylmercury hydroxide or the like, the resulting mixture is spotted as a dot (dot) on a filter such as nitrocellulose at various concentrations to cause hybridization with a probe labeled with a radioactive label or the like and signal intensity is detected. Although there is a possibility of detecting another mRNA having homology with the probe or another mRNA having a length different from the intended mRNA, but it is more convenient than the northern blotting method.

(iii) RNase Protection Assay

This is a method making use of the property of RNase. This nuclease does not degrade a double-stranded RNA that agrees completely with a target RNA and is hybridized thereto, but cleaves at a position of a mismatch, if any. First, a sample RNA is hybridized with an mRNA labeled with ³²P or the like used as a probe. Then, the resulting product is digested with RNase. A reaction product is subjected to electrophoresis on agarose gel, polyacrylamide gel, or the like to determine its size. When a transcription product is completely complementary to the probe RNA, it shows a large band. When there is a mismatch, on the other hand, the band has a decreased size or two or more bands appear, from which presence or absence of the intended RNA can be confirmed.

### (iv) In Situ Hybridization

After a sample obtained from a test subject is pretreated with a proteolytic enzyme or hydrochloric acid, it is blocked with a salmon sperm DNA or albumin in order to suppress non-specific binding of a probe. Then, the tissue sample is hybridized for about 24 hours with a probe RNA labeled with a labeling substance. Then, the tissue sample is washed and a target site is detected by autoradiography or immunohistochemical method. For a trace amount of the target mRNA, in situ RT-PCR is used. A reverse transcription reaction is performed using a reverse transcriptase and an oligo (dT) primer and after amplification of the resulting cDNA by PCR, cDNA is detected using a labeled complementary nucleic acid.

### (v) Real Time PCR

Real time PCR is a method of detecting a PCR amplification product by using fluorescence. It includes two methods: one is an intercalation method using a fluorescence label which is typified by SYBR Green and is specifically inserted into a double-stranded nucleic acid; and a method using a fluorescence-labeled variant-sequence-specific probe typified by TaqMan probe. Also in using real time PCR, a cDNA obtained from the RNA of a sample by using a reverse transcriptase can be used as a template.

### (vi) DNA Microarray

Detection of a variant in RNA can also be achieved using a DNA microarray. A plurality of variants can be detected simultaneously by extracting all the RNAs from the sample of a test subject and using a DNA microarray to which DNAs complementary to RNAs having a plurality of intended variants have been immobilized.

Examples of a method of detecting a variant in a protein include immunoassay by which presence or absence of binding with an antibody is confirmed using an antibody that binds only to either one of CoQ2 having a variant or CoQ2 having no variant without cross reactivity. Such an antibody can be prepared by a method known to those skilled in the art. Detection of binding with an antibody can be performed by labeling an antibody or secondary antibody by a known method. Examples of a labeling substance include enzymes such as peroxidase and alkali phosphatase, radioactive substance such as ¹²⁵I, ¹³¹I, ³⁵S, and ³H, fluorescent substances such as fluorescein isothiocyanate, rhodamine, dansyl chloride, phycoerythrin, tetramethylrhodamine isothiocyanate, and near infrared fluorescent materials, light emitting substances such as luciferase, luciferin, and aequorin, and nano particles such as colloidal gold and quantum dots.

Western blotting is also a method of detecting a variant in a protein. After a sample obtained from a test subject is treated with SDS or the like and is denatured by destroying a protein conformation, the protein is separated by SDS-PAGE based on its molecular weight. After electrophoresis, the gel stacked on a membrane (nitro cellulose, nylon, PVDF, or the like) is set in a transfer apparatus and the protein band in the gel is electrically transferred (blotted) on the membrane. After blocking for preventing non-specific adsorption to a protein, a primary reaction with an antibody specifically binding to CoQ2 having or not having a variant is performed. Then, a secondary antibody labeled with a light emitting enzyme or the like and specifically recognizing a primary antibody molecule is allowed to react with the primary antibody and a target protein is detected through detection of the secondary antibody.

In the present invention, as detection of a variant that deteriorates biosynthesis of CoQ10, deterioration in the function of a CoQ2 protein may be detected.

The deterioration in the function of CoQ2 can be confirmed by measuring reduction in prenylation activity of parahydroxybenzoate. As described in Examples, the CoQ2 activity can be measured by labeling PHB with a radioactive substance and detecting decaprenyl PHB.

### [MSA Risk Test Kit]

The MSA risk test kit according to the present invention includes at least one of the follows (i) to (iii):

(i) a nucleic acid that hybridizes with a region, in a coenzyme Q2 gene, containing a nucleic acid encoding an amino acid at a site selected from the group consisting of position 49, position 57, position 69, position 78, position 97, position 107, position 113, position 267, position 297, position 337, and position 343 of a coenzyme Q2 protein (SEQ ID NO: 1);

(ii) a primer set capable of amplifying a region, in the coenzyme Q2 gene, containing a nucleic acid encoding an amino acid at a site selected from the group consisting of position 49, position 57, position 69, position 78, position 97, position 107, position 113, position 267, position 297, position 337, and position 343 of the coenzyme Q2 protein (SEQ ID NO: 1); and

(iii) an antibody that binds, without cross-reactivity, only to either one of a coenzyme Q2 protein having at least one variant selected from the group consisting of P49H, S57T, R69H, M78V, I97T, P107S, S113F, T267A, S297C, R337Q, R337X, and V343A in SEQ ID NO: 1 or a wild type coenzyme Q2 protein.

These nucleic acids or antibodies can be used for the method for testing risk of MSA according to the present invention.

The nucleic acid (i) can be used for detecting presence or absence of a variant through specific binding to a mutation site of the nucleic acid. The nucleic acid may have, for example, from a base length of from 5 to 100, from 10 to 50, from 15 to 30, or the like. The sequence of the nucleic acid can be designed as needed by those skilled in the art based on the sequence to be detected.

These nucleic acids may be immobilized on a solid phase carrier. The term “solid phase carrier” as used herein is not particularly limited insofar as it is a carrier capable of immobilizing thereon a DNA. Examples include microtiter plates made of glass, metal, or resin, substrates, beads, nitrocellulose membranes, nylon membranes, and PVDF membranes. DNA can be immobilized on such a solid carrier by a known method.

The above-described nucleic acid (ii) allows detection of a variant by specifically amplifying by PCR a nucleic acid having a variant. Each primer may have, for example, from a base length of from 5 to 100, from 10 to 50, from 15 to 30, or the like. The sequence of the primer can be designed as needed by those skilled in the art based on the sequence to be detected.

The antibody (iii) allows detection of a variant of a CoQ2 protein by binding, without cross reactivity, to either one of a variant-having protein or a wild type protein. The antibody can be prepared by a known method by those skilled in the art. The antibody may be labeled in advance or immobilized onto a solid phase carrier. The kit of the present invention may contain a secondary antibody as needed.

The antibody may be either a monoclonal antibody or a polyclonal antibody. The monoclonal antibody can be produced from a hybridoma prepared by isolating an antibody producing cell from an animal immunized with an antigen and fusing the resulting cell with a myeloma cell. The polyclonal antibody can be obtained from the serum or the like of an animal immunized with an antigen. The antibody included in the test kit of the present invention may be labeled in advance with a fluorescent substance, a radioactive substance, or the like.

Such a kit may be equipped with, for example, a detection probe, a reverse transcriptase, various reaction•detection reagents, a buffer, an instruction manual, a secondary antibody, and the like.

### [MSA Diagnostic Method]

The present invention also encompasses an MSA diagnostic method for detecting, from a sample collected from a test subject, a variant that deteriorates biosynthesis of CoQ10. The detection of a variant that deteriorates biosynthesis of CoQ10 can be carried out as in the testing method of the present invention so that a description on it is omitted here.

### [Drug for Prevention or Treatment of MSA, and Preventing or Treatment Method]

A drug for treatment of MSA according to the present invention contains CoQ10 as an effective ingredient thereof. A method of preventing or treating MSA according to the present invention includes a step of administering CoQ10. As described above, a decrease in the amount of CoQ10 due to mutation of an enzyme participating in biosynthesis of CoQ10 may cause MSA.

Administration route to MSA patients is not particularly limited and either oral administration or parenteral administration may be used. Examples of the parenteral administration include administration through injection such as intramuscular injection, intravenous injection, and subcutaneous injection, transdermal administration, and transmucosal administration (nasal, buccal, ocular, pulmonary, vaginal, or rectal) administration.

The CoQ10 may be administered as is or as a preparation obtained by adding thereto a pharmacologically acceptable carrier, an excipient, or an additive. Examples of the dosage form include solutions (for example, injections), dispersions, suspensions, tablets, pills, powders, suppositories, powders, fine granules, granules, capsules, syrups, troches, inhalants, ointments, eye drops, nasal drops, ear drops, and cataplasms.

The preparation can be obtained by the conventional method while using, for example, an excipient, a binder, a disintegrant, a lubricant, a dissolving agent, a solubilizing agent, a colorant, a taste/odor corrigent, a stabilizer, an emulsifier, an absorption promoter, a surfactant, a pH regulator, an antiseptic, and an antioxidant as needed.

Examples of ingredients used for obtaining the preparation include, but not limited to, purified water, saline, a phosphate buffer, pharmacologically acceptable organic solvents such as, dextrose, glycerol, and ethanol, animal and vegetable oils, lactose, mannitol, glucose, sorbitol, crystalline cellulose, hydroxypropyl cellulose, starch, corn starch, silicic anhydride, magnesium aluminum silicate, collagen, polyvinyl alcohol, polyvinylpyrrolidone, carboxyvinyl polymer, sodium carboxymethyl cellulose, sodium polyacrylate, sodium alginate, water-soluble dextran, sodium carboxymethyl starch, pectin, methyl cellulose, ethyl cellulose, xanthan gum, gum arabic, tragacanth, casein, agar, polyethylene glycol, diglycerin, glycerin, propylene glycol, vaseline, paraffin, octyldodecyl myristate, isopropyl myristate, higher alcohol, stearyl alcohol, stearic acid, and human serum albumin.

Pills or tablets may be sugar coated or may be coated with an enteric or gastro-enteric substance.

Injections may contain distilled water for injection, physiological saline, propylene glycol, polyethylene glycol, a vegetable oil, an alcohol, or the like. Furthermore, they may contain a wetting agent, an emulsifier, a dispersant, a stabilizer, a dissolvent agent, a solubilizing agent, an antiseptic, or the like.

CoQ10 may be administered in combination with another drug or treatment method effective against MSA. For example, it can be used in combination with a drug used currently for the symptomatic treatment for MSA. CoQ10 is also useful for the prevention of MSA. When the testing method of the present invention judges that the MSA risk is high, replenishing CoQ10 even when there is no sign of onset can prevent the onset or retard the onset. CoQ10 is contained in a so-called health food and is highly safe and has few side effects so that it can be administered continuously.

When CoQ10 is administered to human patients, its dose is not particularly limited because it differs depending on the age, sex, weight, or susceptibility of the patient, the method of administration, administration interval, the kind of an active ingredient, or the kind of the preparation, but an amount of from 30 MG to 2000 MG, from 40 MG to 1500 MG, or 100 MG to 1200 MG can be administered at once or in several portions.

### [Method for Screening a Drug for Prevention or Treatment of MSA]

The method for screening a drug for prevention or treatment of MSA according to the present invention is a method for screening a drug for the prevention or treatment of multiple-system atrophy. It includes a step of contacting a candidate compound with a cell and a step of selecting the candidate compound that increases the amount of coenzyme Q10 in the cell.

The cell used for the screening method is not particularly limited insofar as it is a cell in which CoQ10 is biosynthesized and examples of it include lymphoblastoid cells.

The candidate compound is also not particularly limited and examples of it may include low molecular compounds, high molecular compounds, nucleic acids, and proteins. Experiment conditions including temperature and time at which the candidate compound is brought into contact with the cell, followed by incubation can be determined as needed by those skilled in the art. For example, it may be a candidate compound enhancing the function of a substance participating in biosynthesis of CoQ10 or a candidate compound inhibiting the function of a substance deteriorating the function of a substance participating in biosynthesis of CoQ10.

Whether the amount of CoQ10 in the cell increases or not can also be found in a known manner by those skilled in the art. The amount may be evaluated by measuring the CoQ10 activity.

### [Nucleic Acid Encoding CoQ2 Protein]

The V343A variant was exclusively found in Japanese people so study on it and elucidation of the onset mechanism are presumed to be useful for the research and development of a method for treating or preventing MSA. A nucleic acid containing V343A variant is useful for such a research and development. The present invention further encompasses a recombinant vector containing such a nucleic acid and a transformant containing the vector.

Disclosure of all the patent documents and non-patent documents cited herein is incorporated herein by reference in its entirety.

### EXAMPLES

The present invention will hereinafter be described specifically based on Examples, but the present invention is not limited to or by them. The present invention can be changed to various modes by those skilled in the art without departing from the significance of the present invention and such a change is encompassed within the scope of the present invention.

### 1. Subject and Method

1-1. Approval of Study with Humans as Subject

Test subjects were registered in the research program approved by the review board of the University of Tokyo and other participating research institutes. Written informed consent was obtained from all the test subjects.

### 1-2. MSA Multiplex Family

Diagnosis of MSA was given based on the diagnostic criteria of MSA on which a consensus has been formed (Gilman S, et al. Neurology 2008; 71: 670-6).

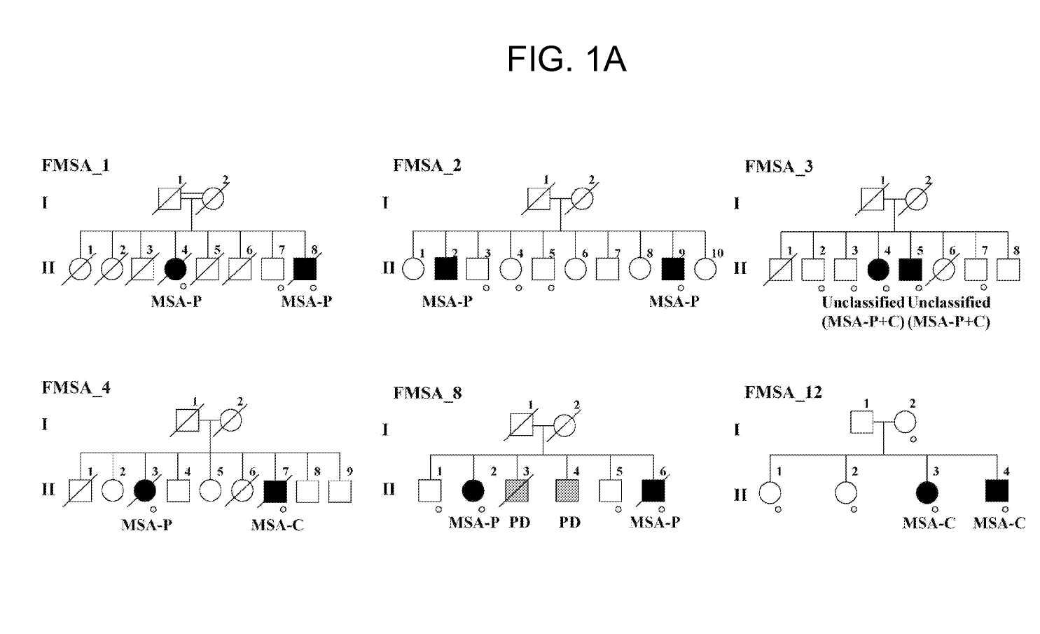

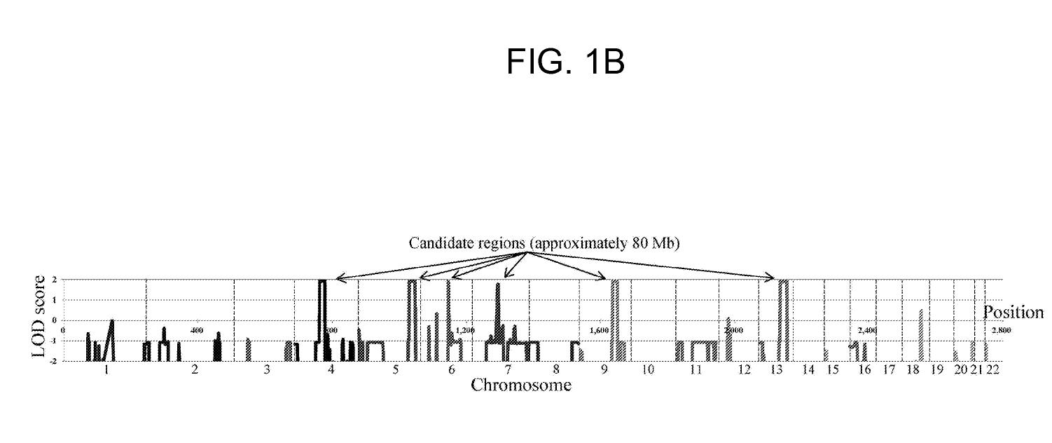

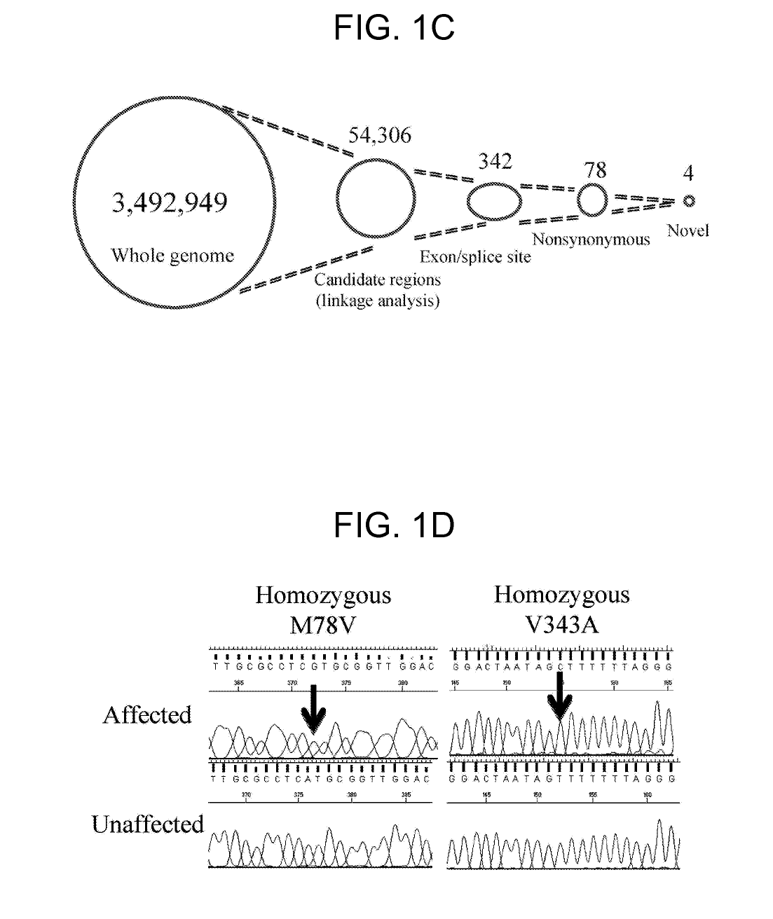

The present inventors heretofore reported four Japanese MSA multiplex families (from FMSA_{—}1 to FMSA_{—}4) (Document 12). Six MSA multiplex families including two new Japanese MSA families (MSA_{—}8 and FMSA_{—}12) having two pairs of brothers suffering from MSA registered in the present study (FIG. 1A). FMSA_{—}1 includes consanguineous marriage (parents are first degree cousins), suggesting the possibility of autosomal recessive inheritance. Clinical findings of the six MSA multiplex families are shown in Table 2. II-4 and II-8 of FMSA_{—}1 and II-6 of FMSA_{—}8 were subjected to biopsy and results were used for definite diagnosis of MSA.

TABLE 2

| | FMSA_1 | | FMSA_2 | | FMSA_3 | | FMSA_4 | | FMSA_8 | | FMSA_12 | |
|---|---|---|---|---|---|---|---|---|---|---|---|---|
| | II-4 | II-8 | II-2 | II-9 | II-4 | II-5 | II-3 | II-7 | II-2 | II-6 | II-3 | II-4 |
| Sex | Female | Male | Male | Male | Female | Male | Female | Male | Female | Male | Female | Male |
| Age at | 68 | 62 | 72 | 63 | 68 | 67 | 69 | 58 | 53 | 52 | 50 | 44 |
| onset, | | | | | | | | | | | | |
| year | | | | | | | | | | | | |
| Age at | 71 | 66 | 73 | 66 | 72 | 68 | 72 | 63 | 76 | 63 | 61 | ? |
| examin- | | | | | | | | | | | | |
| ation, | | | | | | | | | | | | |
| year | | | | | | | | | | | | |
| Initial | Tremor | Ataxia | Tremor | Tremor | Tremor | Ataxia | Akinesia | Impotence | Akinesia | Akinesia | Ataxia | Ataxia |
| symptoms | | | | | | | | | | | | |
| Parkin- | + | + | + | + | + | + | + | + | + | + | + | + |
| sonism | | | | | | | | | | | | |
| Cerebellar | + | + | − | + | + | + | − | + | − | − | + | + |
| sign | | | | | | | | | | | | |
| Enhanced | + | − | − | − | − | − | + | + | − | + | − | + |
| muscle | | | | | | | | | | | | |
| stretch | | | | | | | | | | | | |
| reflexes | | | | | | | | | | | | |
| Babinski | − | − | − | − | − | − | + | + | − | − | − | − |
| sign | | | | | | | | | | | | |
| Urinary | + | + | + | + | − | + | + | + | + | + | + | + |
| dys- | | | | | | | | | | | | |
| function | | | | | | | | | | | | |
| Orthostatic | − | − | + | + | + | + | + | + | N.E. | + | + | − |
| hypo- | | | | | | | | | | | | |
| tension | | | | | | | | | | | | |
| Response | Poor | Poor | Poor | Poor | Poor | Poor | Poor | Poor | Poor | Poor | N.E. | N.E. |
| to | | | | | | | | | | | | |
| levodopa | | | | | | | | | | | | |
| Cerebellar | ? | + | − | + | − | + | + | + | − | + | + | + |
| atrophy | | | | | | | | | | | | |
| on brain | | | | | | | | | | | | |
| MRI | | | | | | | | | | | | |
| Pontine | ? | + | − | + | − | + | + | + | + | + | + | + |
| atrophy | | | | | | | | | | | | |
| on brain | | | | | | | | | | | | |
| MRI | | | | | | | | | | | | |
| Cross sign | ? | + | − | + | − | + | − | + | − | + | + | + |
| on brain | | | | | | | | | | | | |
| MRI | | | | | | | | | | | | |
| Slitlike | ? | + | + | − | − | − | + | + | + | − | + | − |
| signal | | | | | | | | | | | | |
| change | | | | | | | | | | | | |
| at the | | | | | | | | | | | | |
| putaminal | | | | | | | | | | | | |
| margin | | | | | | | | | | | | |
| on brain | | | | | | | | | | | | |
| MRI | | | | | | | | | | | | |
| Criteria by | Definite | Definite | Probable | Probable | Probable | Probable | Probable | Probable | Probable | Definite | Probable | Probable |
| Gilman⁴ | | | | | | | | | | | | |
| Pheno- | MSA-P | MSA-P | MSA-P | MSA-P | MSA-P + C | MSA-P + | MSA-P | MSA-C | MSA-P | MSA-P | MSA-C | MSA-C |
| types | | | | | | C | | | | | | |
| Compli- | Retinitis | Retinitis | — | — | Rheumatoid | — | — | — | Cerebral | — | — | — |
| cation | Pigmentosa | Pigmentosa | | | arthritis | | | | infarction | | | |

Abbreviations:
+, present;
−, absent;
?, unknown;
N.E., not evaluated;
MRI, magnetic resonance imaging;
MSA-C, multiple system atrophy of the cerebellar type;
MSA-P, multiple system atrophy with predominant parkinsonism.

### 1-3. Sporadic MSA Patients and Controls

The diagnosis of MSA was given in accordance with the criteria on which consensus had been formed (Gilman S, et al. Neurology 2008; 71: 670-6). A Japanese cohort includes 195 MSA patients and 113 healthy subjects, samples of which were provided by the Japan Multiple System Atrophy Research Consortium (JAMSAC). Further, 168 MSA patients and 407 control subjects from the University of Tokyo, Brain Bank for Aging Research, Tokyo Metropolitan Geriatric Hospital and Institute of Gerontology, Brain Research Institute/Niigata University, Hokkaido University Graduate School of Medicine, and Kagoshima University Graduate School of Medical and Dental Sciences were used for diagnosis.

As an independent MSA cohort, European and North American cohorts were used. European genomic DNA samples included those from 138 MSA patients and 281 control subjects in Pitie-Pitié-Salpêtrière Hospital (France), 34 MSA patients and 34 control subjects in University of Federico II (Italy), and 46 MSA patients in University of Bonn (Germany). The European cohort included five MSA patients in the University of Sydney. The North American genomic DNA samples included those from 172 MSA patients and 294 control subjects provided by North American Multiple System Atrophy Study Group (NAMSA-SG). The statistics of the participants are shown in Table 3.

TABLE 3

| | | | | | | Phenotype |
|---|---|---|---|---|---|---|
| | | | | | | (MSA-C/ |
| | Ethnic | | Age at | Age at | Male/ | MSA-P/ |
| | series | Number | sampling | onset | Female | Unclassified) |
| MSA | Japan | 363 | 61.4 | 59.5, | 211/152 | 259/85/19 |
| patients | | | (8.5) | 8.6 | | |
| | Europe | 223 | 60.0 | 55.4, | 138/85 | 191/22/10 |
| | | | (7.9) | 8.3 | | |
| | North | 172 | N.D. | 58.4, | 103/69 | 52/107/13 |
| | America | | | 9.5 | | |
| Control | Japan | 520 | 68.7 | N.A. | 255/265 | N.A. |
| Subjects | | | (11.0) | | | |
| | Europe | 315 | 58.9 | N.A. | 150/165 | N.A. |
| | | | (6.1) | | | |
| | North | 294 | 65.2 | N.A. | 156/138 | N.A. |
| | America | | (9.0) | | | |

Abbreviations:
MSA-C, multiple system atrophy of the cerebellar type;
MSA-P, multiple system atrophy with predominant parkinsonism;
N.A., not applicable;
N.D., not described.
Age at sampling and age at onset are presented as mean, standard deviation.

### 1-4. Independent Cohort of Control Subjects and Cohort of Other Neurodegenerative Diseases

Independent cohort (n=2,383) of control subjects for replication study was provided by Japanese Genetic Study Consortium for Alzheimer Disease (JGSCAD) and Japanese Consortium for Amyotrophic Lateral Sclerosis research (JaCALS). In order to study the specificity of the relation between a CoQ2 variant and MSA, also surveyed were other neurodegenerative diseases (Alzheimer's disease (AD), Parkinson disease (PD), and amyotrophic lateral sclerosis (ALS) patients; 2,728 AD patients from JGSCAD, 659 PD patients from the University of Tokyo and Japanese Parkinson Disease Susceptibility Gene Consortium, and 634 ALS patients from the University of Tokyo and JaCALS).

### 1-5. Relation Analysis and Whole Genome Sequencing

Relation analysis was carried out for FMSA_{—}1 by using Affymetrix SNP 6.0 arrays. The genomic DNA sample of the patient II-4 in FMSA_{—}1 was analyzed four times using Illumina Genome Analyzer IIx (100-bp-long paired ends). For detection of alignment and mutation with respect to the human genome reference sequence (NCBI36/hg18 assembly), Burrows Wheeler Aligner (BWA) and Smatools were used.

### 1-6. Mutation Analysis of COQ2 Gene

PCR was performed using a primer pair listed in Table 4 that amplifies each of exons of the CoQ2 gene, followed by nucleotide sequence analysis.

TABLE 4

| Primer | Forward primer sequence | Reverse primer sequence |
|---|---|---|
| Exon 1 | 5′-TGAAGGAGGGCCACGAGAA-3′ | 5'-CCTAGAGTAAGCGACCACGATG-3′ |
| Exon 2 | 5′-GGGGTCCTTTGTGATTTGAG-3′ | 5′-TTCCATGCTGGATTTCTGTG-3′ |
| Exon 3 | 5′-TACCATGGGCCAGTCTCTTC-3′ | 5'-TGTGTGGTGAGTTACTTACACTTGC-3′ |
| Exon 4 | 5′-TTGTCTTAAAGTATTTCGTGGTTTC-3′ | 5'-ATCTCTCCATAAAAGTGTAGTTTGC-3′ |
| Exon 5 | 5′-CACTGAACACACTCCGATGC-3′ | 5′-TGCTTTCTCCTTAATTTGGTTC-3′ |
| Exon 6 | 5′-TCACCGCTTATGGTATATCTGC-3′ | 5'-TGCCAGGTAAACACAGAGGG-3′ |
| Exon 7 | 5′-TTTGCTGTTTTCTCCTCCG-3′ | 5′-AAATCTTCATCTTCAGGTTCTTAATTC-3′ |

Polymerase chain reaction (PCR) was performed using primer pairs to amplify each exon of COQ2 with LATaq (TaKaRa). Direct nucleotide sequence analysis was performed using ExoSAP-IT (USB, OH, USA), a BigDye Terminator v3.1 kit, and XTerminator using ABI 3130 and 3730 Genetic Analyzers (Life Technologies, CA, USA).

Exon 1 Forward primer sequence SEQ ID NO: 2
Exon 1 Reverse primer sequence SEQ ID NO: 3
Exon 2 Forward primer sequence SEQ ID NO: 4
Exon 2 Reverse primer sequence SEQ ID NO: 5
Exon 3 Forward primer sequence SEQ ID NO: 6
Exon 3 Reverse primer sequence SEQ ID NO: 7
Exon 4 Forward primer sequence SEQ ID NO: 8
Exon 4 Reverse primer sequence SEQ ID NO: 9
Exon 5 Forward primer sequence SEQ ID NO: 10
Exon 5 Reverse primer sequence SEQ ID NO: 11
Exon 6 Forward primer sequence SEQ ID NO: 12
Exon 6 Reverse primer sequence SEQ ID NO: 13
Exon 7 Forward primer sequence SEQ ID NO: 14
Exon 7 Reverse primer sequence SEQ ID NO: 15

### 1-7. Functional Analysis of COQ2 Gene by Yeast Complementation System

Site-directed mutagenesis of a wild type human COQ2 gene was carried out by PCR while using primers (SEQ ID NOS: 16 to 47 from the top) listed in Table 5. Then, wild type and mutated human COQ2 gene cDNAs were each inserted into a yeast expression type pAUR123 vector (product of Takara). A BY4741Δcoq2 strain, that is, a yeast coq2 gene null variant was transformed with the pAUR123 vector containing the wild type or mutated human COQ2 gene cDNA by using Yeastmaker Yeast Transformation System 2 (product of Clontech). Proliferation in a medium containing a non-fermentable carbon source (yeast extract•peptone•glycerol medium) was measured by monitoring the 600-nm absorbance (OD600) of the medium through an OD monitor (product of Titech).

TABLE 5

| Primer | Sequence |
|---|---|
| L16V-F | 5′-TCGCGCGGGGCCTGCGGGCTGTGGCACTGGC-3′ |
| L16V-R | 5′-AGCCCGCAGGCCCCGCGCGAACCCCGCGG-3′ |
| P22L-F | 5′-TGTGGCACTGGCGTGGCTGCTGGGCTGGCGGG-3′ |
| P22L-R | 5′-GCAGCCACGCCAGTGCCACAGCCCGCAGGC-3′ |
| F29L-F | 5′-CGGGCTGGCGGGGCCGCTCCCTCGCCCTGGCG-3′ |
| F29L-R | 5′-GGAGCGGCCCCGCCAGCCCGGCAGCCACGC-3′ |
| P49H-F | 5′-TTGCAGCCCCCCGCCTGTCACGAGCCGCGC-3′ |
| P49H-R | 5′-GACAGGCGGGGGGCTGCAAGTCACCACGT-3′ |
| S57T-F | 5′-GCCGCGCGGGCGCCAGCTCACTTTGTCCGCGG-3′ |
| S57T-R | 5′-TGAGCTGGCGCCCGCGCGGCTCGGGACAGG-3′ |
| R69H-F | 5′-GGTGGTGGACTCTGCGCCCCACCCCCTGCAG-3′ |
| R69H-R | 5′-GGGGCGCAGAGTCCACCACCGCCGCCGCGG-3′ |
| M78V-F | 5′-TGCAGCCGTACTTGCGCCTCGTGCGGTTGGAC-3′ |
| M78V-R | 5′-GAGGCGCAAGTACGGCTGCAGGGGGCGGGG-3′ |
| I97T-F | 5′-TTTACCATGTACCTGGAGCACTGGTTTGGCAG-3′ |
| I97T-R | 5′-TGCTCCAGGTACATGGTAAATACAGAAGCC-3′ |
| P107S-F | 5′-CAGCTGAACCAGGTTGTTTTTCAGATTGGTAC-3′ |
| P107S-R | 5′-AAAACAACCTGGTTCAGCTGCCAAACCAT-3′ |
| S113F-F | 5′-TCCAGATTGGTACATGCTCTTCCTCTTTGGCA-3′ |
| S113F-R | 5′-AGAGCATGTACCAATCTGGAAAACAACCT-3′ |
| T267A-F | 5′-TTTTGATTGGTCTTAAGTCAGCGGCTCTGCGG-3′ |
| T267A-R | 5′-TGACTTAAGACCAATCAAAACATCATCTCT-3′ |
| S297C-F | 5′-TGAGCCTAGTGGGTGTGAACTGTGGACAGACT-3′ |
| S297C-R | 5′-GTTCACACCCACTAGGCTCAGTGCCCCCAG-3′ |
| N336H-F | 5′-GTTGGAATAAATTTATCTCCCACCGAACACTG-3′ |
| N336H-R | 5′-GGAGATAAATTTATTCCAACAATCCTCAGG-3′ |
| R337Q-F | 5′-GAATAAATTTATCTCCAACCAAACACTGGGAC-3′ |
| R337Q-R | 5′-GGTTGGAGATAAATTTATTCCAACAATCCT-3′ |
| R337X-F | 5′-GGAATAAATTTATCTCCAACTGAACACTGGGA-3′ |
| R337X-R | 5′-GTTGGAGATAAATTTATTCCAACAATCCTC-3' |
| V343A-F | 5′-CCGAACACTGGGACTAATAGCTTTTTTAGGG-3′ |
| V343A-R | 5′-CTATTAGTCCCAGTGTTCGGTTGGAGATAA-3' |

PCR-based site-directed mutagenesis of wild-type human COQ2 was carried out using the primers with a QuickChange Site-Directed Mutagenesis kit (Stratagene, CA, USA).

### 1-8. Measurement of CoQ2 Activity

CoQ2 (EC 2.5.1.39) activity was assayed by measuring the incorporation of radioactive parahydroxybenzoate (PHB) into decaprenyl PHB. More specifically, a mitochondrial fraction prepared from lymphoblastoid cells using QProteome Mitochondria Isolation kit (product of Qiagen) was used as an enzyme source. In accordance with the method of Lopez-Martin, et al (Lopez-Martin J M, et al. Hum Mol Genet 2007; 16: 1091-7), a reaction mixture was prepared, which was composed of a 500 μg mitochondria-rich fraction, 1000 μM [¹⁴C] PHB (1.85 MBq/μmol), and a 100 μL assay buffer containing 5 μM decaprenyl pyrophosphate (containing 0.05% 3-[(3-chloramidopropyl)dimethylammonio]-1-propanesulfonate (CHAPS)). The reaction was made at 37° C. for 60 minutes, followed by extraction with 1000 μL hexane. The radioactive substance in the hexane phase was measured using a liquid scintillation counter Tri-Garb 2000CA (product of PerkinElmer). The results are shown by the mean of nine independent experiments.

### 1-9. Measurement of CoQ10 Level in the Tissue

EB virus immortalized lymphoblastoid cells (provided by JAMSAC) established from 152 MSA patients and 76 control subjects were cultured in a RPMI-1640 medium containing 10% fetal calf serum. The free cholesterol and CoQ10 were extracted from about 10⁷ to 10⁸ lymphoblastoid cells with 4 times the amount of 2-propanol or from a frozen cerebral sample prepared by the biopsy of three MSA patients and three control subjects with 9 times the amount of 2-isopropanol.

The free cholesterol concentration and total CoQ10 concentration (ubiquinone 10 and ubiquinol 10) in the extract were measured using a high-performance liquid chromatography.

### 1-10. Statistical Analysis

All the results were indicated by mean±standard deviation. A significant difference of the mean age at onset in carriers and non-carriers was evaluated by Student's t-test; a significant difference of the allele frequency and contingency table was evaluated by the Yates correction chi-square test or Fisher's exact test; and 95% confidence interval (CI) corresponding to an odds ratio was evaluated by Fisher's exact test. For group comparison, Kruskal-Wallis test and Steel method were used. All the statistics were performed on two-sided test and the level of significance was set at p=0.05.

### 2. Results

### 2-1. Identification of Causative Gene of Familial MSA

According to the parametric multipoint linkage analysis of FMSA_{—}1 using an autosomal recessive inheritance system genetic model, the maximum LOD score in the 80-Mb region including chromosomes 4, 5, 6, 7, 9, and 13 was 1.93 (FIG. 1B).

Whole genome sequencing of a sample obtained from the patient (II-4) in FMSA_{—}1 generated 187.5 Gb of short reads in total. An average coverage of the reference genome was 58 times. The mutation candidates of the above family were narrowed down to four novel non-synonymous SNVs by starting with single nucleotide variants (SNVs) or insertion/deletions of 47,830 located in the candidate region (FIG. 1C).

The four SNVs were c.2120A>G, p.K707R in SHROOM3 gene (NM_{—}020859, Q8TF72), c.1178T>C, p.V343A in COQ2 gene (NM_{—}015697, Q96H96), c.382A>G, p.M78V in COQ2 gene, and c.691A>G, p.R231G in SCEL gene (NM_{—}144777, 095171).

As a result of screening 180 Japanese control samples, only M78V of the COQ2 gene was not found in them. The allele frequencies of K706R in the SHROOM3 gene, V343A in the COQ2 gene, and R231 G in the SCEL gene were 3/360, 5/360, and 98/360, respectively. Since familial MSA was very rare, the present inventors thought there was a high possibility of M78V, a variant of the COQ2 gene encoding parahydroxybenzoate•polyprenyl transferase involved in the biosynthesis of CoQ10 being a cause responsible for familial MSA autosomal recessive inheritance. Simultaneous isolation analysis of FMSA_{—}1 has revealed that two patients (II-4 and II-8) carried the homozygous M78V and V343A in the COQ2 gene and the unaffected brother (II-7) carried a wild type sequence (FIG. 1D).

Based on these findings, nucleotide sequences in the code region of the COQ2 gene and a splice region contiguous thereto in the other plurality of MSA families were subjected to direct analysis. In addition, a heterozygous variant composed of nonsense (c.1159C>T, p.R337X) variants and missense variants (c.1178T>C, p.V343A) in the CoQ2 gene were found in the affected brothers (II-3 and II-4) in FMSA_{—}12. Simultaneous isolation analysis of FMSA_{—}12 has revealed that their mother (I-2) was heterozygous for V343A, unaffected brother (II-1) had a wild type sequence, and the other unaffected brother (II-2) was heterozygous for R337X. It has therefore been confirmed that two affected brothers (II-3 and II-4) were compound-heterozygous for R337X and V343A. R337X was not observed in the 180 Japanese controls. In the other four MSA families (FMSA_{—}2, FMSA_{—}3, FMSA_{—}4, and FMSA_{—}8), mutation of the COQ2 gene was not detected.

The above-described results have revealed that in the two families with familial MSA, FMSA_{—}1 and FMSA_{—}12, onset of MSA is triggered with the presence of homozygous M78V and V343A or compound heterozygous R337X and V343A as a sufficient condition.

2-2. Association of CoQ2 Variant with Sporadic MSA

In order to study the participation of the COQ2 gene mutation in sporadic MSA, resequencing of the COQ2 gene of a greater Japanese cohort (363 MSA patients and 520 control subjects) was carried out. Although only one nonsynonymous COQ2 gene variant (L16V, rs6818847) was registered in dbSNP130, the present inventors confirmed that the allele frequency of L16V of the Japanese MSA patients was 0.90 and that of the Japanese control subjects was 0.88 so that the variant was not included in an object to be analyzed later.

It has been elucidated that as a result of the resequencing of the COQ2 gene, four MSA patients had two variants simultaneously (one had I97T/V343A, one had R337Q/V343A, and two had V343A/V343A) but none of the controls had two variants in the COQ2 gene (Table 6).

TABLE 6

| | Japanese series | | European series | | North American series | |
|---|---|---|---|---|---|---|
| | MSA | Control | MSA | Control | MSA | Control |
| | Patients | Subjects | Patients | Subjects | Patients | Subjects |
| Genotypes | n = 363 | n = 520 | n = 223 | n = 315 | n = 172 | n = 294 |
| P22L/wt | 0 | 1 | 0 | 0 | 0 | 0 |
| F29L/wt | 0 | 0 | 1 | 0 | 0 | 0 |
| P49H**/wt | 0 | 0 | 0 | 0 | 1 | 0 |
| S57T**/wt | 0 | 0 | 1 | 0 | 0 | 0 |
| R69H**/wt | 0 | 0 | 0 | 0 | 0 | 1 |
| I97T*, | 1 | 0 | 0 | 0 | 0 | 0 |
| V343A§ | | | | | | |
| P107S**/wt | 1 | 0 | 0 | 0 | 0 | 0 |
| S113F**/wt | 1 | 0 | 0 | 0 | 0 | 0 |
| T267A*/wt | 0 | 0 | 1 | 0 | 0 | 0 |
| S297C*/wt | 0 | 0 | 1 | 0 | 0 | 0 |
| N336H/wt | 0 | 1 | 0 | 0 | 0 | 0 |
| R337Q**/ | 1 | 0 | 0 | 0 | 0 | 0 |
| V343A§ | | | | | | |
| V343A§/wt | 29 | 17 | 0 | 0 | 0 | 0 |
| V343A§/ | 2 | 0 | 0 | 0 | 0 | 0 |
| V343A§ | | | | | | |

Abbreviations:
wt, wild-type sequence;
MSA, multiple system atrophy;
*mildly or **severely deleterious variants identified using the yeast complementation system;
§, decreased COQ2 activity determined by enzyme assay.

The nucleotide sequence analysis of subcloned mutated alleles has revealed that the R337Q/V343A was compound heterozygous. Distance between I97T and V343A was too large to be amplified by PCR and the genomic DNA sample from parents was not obtained, making it impossible to determine the phase of I97T/V343A. It has been confirmed that 29 MSA patients were heterozygous for V343A and two MSA patients had respectively different heterozygous variants (P107S and S113F), but it has also been confirmed that 17 control subjects were heterozygous for V343A and two control subjects had different heterozygous variants (P22L and N336H).

Of the COQ2 gene variants, V343A was relatively common in the Japanese people. As shown in Table 7, it has been confirmed that the allele frequency of V343A is 35/726 (4.8%) in the Japanese MSA patients and 17/1,040 (1.6%) in the Japanese control subjects; and that an odds ratio for the MSA patients compared with the control subjects is 3.05 (95% C.I., from 1.65 to 5.85) and is significant (p=1.5×10⁻⁴). Further, genotyping of V343A was performed in the second cohort (n=2,383) of the Japanese control subjects, revealing that the allele frequency of V343A in the control subjects is 106/4,766 (2.2%) and an odds ratio for the MSA patients compared with the second cohort of the control subjects is 2.23 (95% C.I., from 1.46 to 3.32, p=6.0×10⁻⁵).

TABLE 7
Association of V343A with MSA in Japanese series

| | MSA patients and control subjects | | Patients with other neurological diseases | | | |
|---|---|---|---|---|---|---|
| | Japanese series | | Japanese series | | | |
| | | Control subjects | | | ALS | Control subjects |
| | MSA patients | (Tier 1) | AD patients | PD patients | patients | (Tier 2) |
| Genotype | n = 363 | n = 520 | n = 2,728 | n = 659 | n = 634 | n = 2,383 |
| Heterozygous V343A | 31 | 17 | 105 | 33 | 31 | 106 |
| Homozygous V343A | 2 | 0 | 2 | 0 | 0 | 0 |
| Allele frequency of | 35/726 | 17/1040 | 109/5,456 | 33/1,318 | 31/1.268 | 106/4,766 |
| V343A | (4.8%) | (1.6%) | (2.0%) | (2.5%) | (2.4%) | (2.2%) |
| Odds ratio (95% C.I.) | 3.05 (1.65-5.85) | | | | | |
| compared with tier 1 | p = 1.5 × 10⁻⁴ | | | | | |
| Odds ratio (95% C.I.) | | | 2.23 (1.46-3.32) | | | |
| compared with tier 2 | | | p = 6.0 × 10⁻⁵ | | | |

Association of functionally deleterious variants* with MSA in combined series

| | | | Allele frequency | Allele frequency | | |
|---|---|---|---|---|---|---|
| | | Number of | of functionally | of functionally | | Fisher |
| | Number of | controls | deleterious variant | deleterious variants | Odds ratio | exact |
| | MSA patients | subjects | in msa patients | in control subjects | (95% C.I.) | test |
| Combined series | 758 | 1,129 | 8/1,516 | 1/2,258 | 11.97 | p = 0.0039 |
| (Japanese, European | | | (0.53%) | (0.05%) | (1.60-531.5) | |
| and North American) | | | | | | |

Abbreviations:
wt, wild-type sequence;
MSA, multiple system atrophy;
AD, Alzheimer disease;
PD, Parkinson disease;
ALS, amyotrophic lateral sclerosis;
C.I., confident interval.
*Functionally deleterious variants are P49H, S57T, R69H, I97T, P107S, S113F, T267A, S297C, and R337Q as determined by the yeast complementation assay.

As a result of genotyping of other neurological diseases including AD, PD and ALS, the allele frequency of V343A in AD patients was 109/5,456 (2.0%) (two of them had homozygous V343A), that in PD patients is 33/1,318 (2.5%), and that in ALS patients is 31/1268 (2.4%). No significant difference is found in the allele frequency between the first cohort and the second cohort of the control subjects and specificity of the COQ2 gene having a V343A variant to MSA has been confirmed.

Next, the MSA cohort of European and American series was analyzed. In the European cohort, four singleton variants (F29L, S57T, T267A, and S297C) were found in each MSA patient, but there was no control subject having a CoQ2 variant (Table 6). In the North American cohort, one singleton variant (P49H) was found in one MSA patient and also one singleton variant (R69H) was found in one control subject (Table 6). It is interesting that in the European and American cohort, in neither MSA patient group nor control subject group, the V343A variant relatively frequently found in the Japanese series was found. Since variants other than V343A were rarely found in either cohort (Table 6), relation between these variants and MSA was studied using these cohorts in combination, while paying attention to the association of COQ2 gene mutation with functional disorder.

### 2-3. Functional Analysis of COQ2 Gene Variant by Yeast Complementary Assay

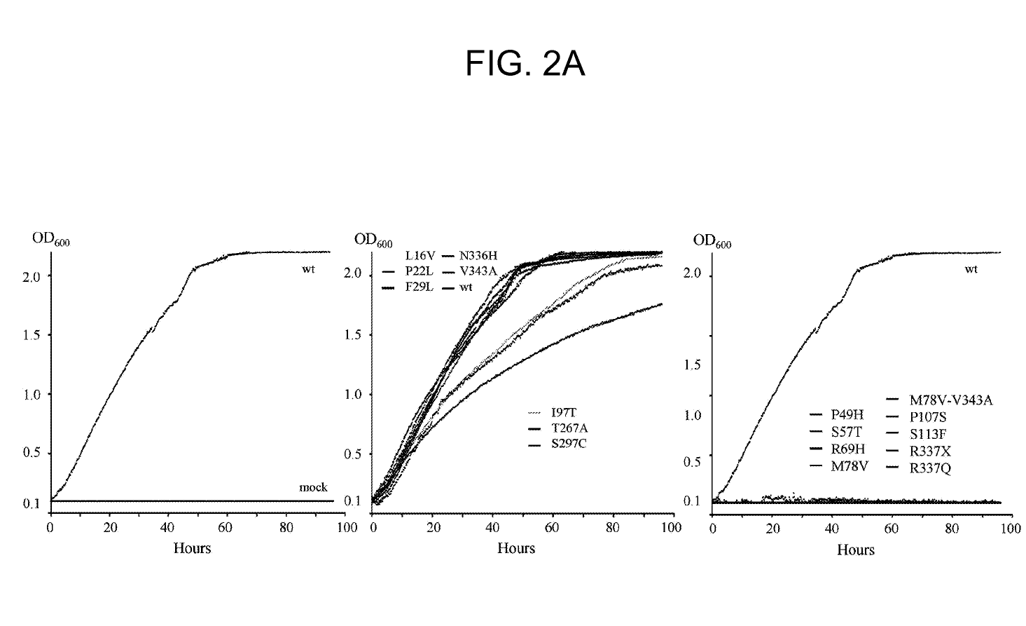

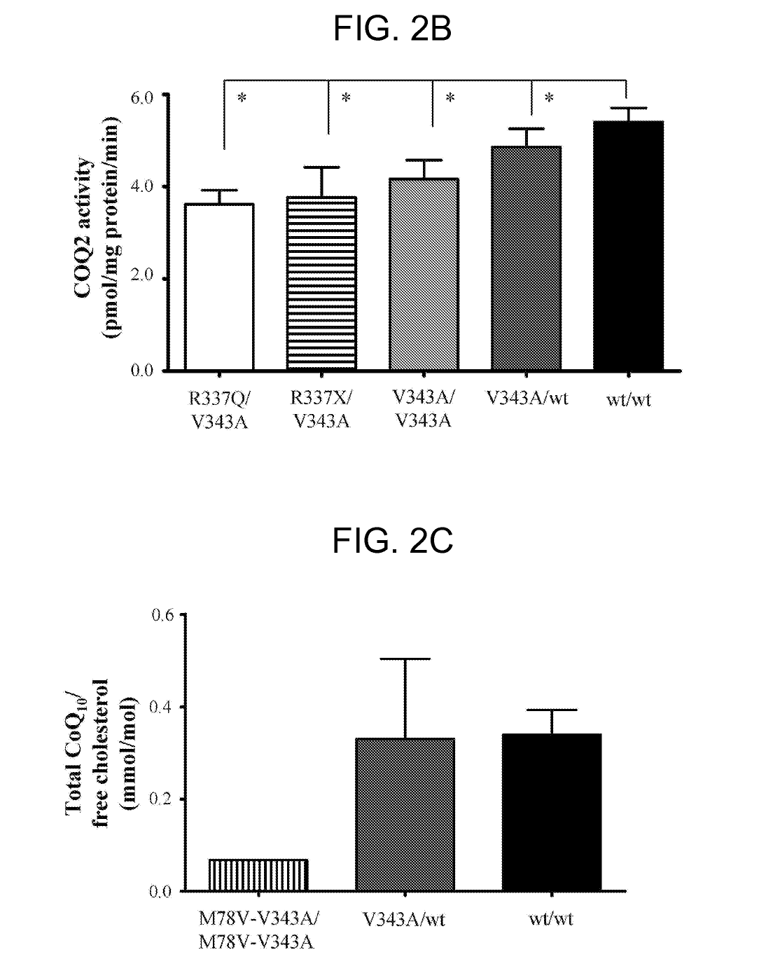

In order to study the function effect of each variant on the CoQ10 biosynthesis and aerobic energy production, a yeast coq2 gene null variant was transformed with wild-type or mutated human COQ2 gene cDNA and functional complementary analysis was performed (FIG. 2A). In the transformant with the mutated COQ2 gene (having P49H, S57T, R69H, M78V, M78V-V343A, P107S, S113F, R337Q, and R337X) of a BY4741Δcoq2 yeast strain, respiration-dependent growth showed a marked reduction as was found in the coq2 null strain (very deleterious variants). Further, the transformant with mutated COQ2 cDNA (having I97T, T267A, and S297C) showed a growth rate much lower than the transformant expressing the wild type CoQ2 but higher than that of the coq2 null strain (mildly deleterious variants). The transformant with the COQ2 cDNA (having L16V, P22L, F29L, N336H, and V343A) showed a growth rate equal to that of the transformant expressing the wild type CoQ2.

Paying attention to rare variants identified in a patient-control subject cohort, it has been recognized in the yeast complementation assay that nine variants (P49H, S57T, R69H, I97T, P107S, S113F, T267A, S297C, and R337Q) were mildly or severely deleterious. By the analysis of these functionally deleterious rare variants in combined three cohorts, eight variants (P49H, S57T, I97T, P107S, S113F, T267A, S297C, and R337Q) were identified in the MSA cohort (n=758) and only one variant (R69H) was identified in the control subject cohort (n=927). An odds ratio of the allele frequency of the deleterious variants in the MSA patients compared with that in the controls was 9.83 (95% C.I., 1.31 to 436.4). It was significant (p=0.014) (Table 7).

### 2-4. CoQ2 Activity in Lymphoblastoid Cells

Next, CoQ2 activity in available lymphoblastoid cells having COQ2 variants was measured in association with the V343A variant. V343A is a variant closely associated with MSA. This variant was selected because it showed normal growth in the yeast complementation assay. The CoQ2 activity in lymphoblastoid cells obtained from MSA patients having any of the following CoQ2 variants (R337Q/V343A, R337X/V343A, V343A/V343A, and V343A/wt) and that in lymphoblastoid cells obtained from controls having no variant were measured. The CoQ2 activity of those patients was markedly lower than that of the controls having no variant (FIG. 2B). The CoQ2 activity in the V343A variant showed a marked decrease, though the yeast coq2 null strain obtained by transformation with V343A mutated COQ2 cDNA showed a normal growth rate in yeast complementation analysis.

### 2-5. Correlation Between Genotype and Phenotype

Table 8 shows clinical characteristics of sporadic MSA patients who are carriers of COQ2 gene mutation (functionally impaired CoQ2 confirmed in yeast complementation assay and CoQ2 activity measurement) and those of non-carrier sporadic MSA patients.

TABLE 8

| | | | | | | Phenolype |
|---|---|---|---|---|---|---|
| | | | | | | (MSA-C/ |
| | | | | Age at | Male/ | MSA-P/ |
| Category | Genotype | Number | Cohort | onset | Female | Unclassified) |
| Carriers | All variants | 39 | All | 61.7, 8.2 | 27/12 | 34/5/0* |
| | P49H/wt | 1 | North America | 61 | 1/0 | 0/1/0 |
| | S57T/wt | 1 | Europe | 50 | 0/1 | 1/0/0 |
| | I97T, V343A | 1 | Japan | 57 | 1/0 | 1/0/0 |
| | P107S/wt | 1 | Japan | 57 | 1/0 | 1/0/0 |
| | S113F/wt | 1 | Japan | 60 | 1/0 | 1/0/0 |
| | T267A/wt | 1 | Europe | 66 | 1/0 | 1/0/0 |
| | S297C/wt | 1 | Europe | 54 | 1/0 | 1/0/0 |
| | R337Q/V343A | 1 | Japan | 61 | 1/0 | 1/0/0 |
| | V343A/wt | 29 | Japan | 62.8, 9.0 | 20/9 | 25/4/0 |
| | V343A/V343A | 2 | Japan | 61.0, 1.4 | 0/2 | 2/0/0 |
| Noncarriers | wt/wt | 719 | All | 57.3, 8.7 | 425/294 | 468/209/42 |

Abbreviations:
wt, wild-type sequence;
MSA-C, multiple system atrophy of the cerebellar type;
MSA-P, multiple system atrop predominant parkinsonism.
Age at sampling and age at onset are preserved as mean, standard deviation.
*The ratio of MSA-C to MSA-P was significantly higher in carriers of COQ2 variants than in noncarriers, as determined by th Fisher exact test using the 2 × 2 contingency table.

The mean age of carriers at onset of MSA was higher than that of noncarriers and a difference between them was significant (p=0.0021). As the phenotype, 34 carriers had MSA-C, 5 carriers had MSA-P, one of the carriers had an unclassified type, 468 noncarriers had MSA-C, 209 noncarriers had MSA-P, and 42 noncarriers had an unclassified type. A ratio of the number of patients with MSA-C to the number patients with MSA-P as determined by the Fisher's exact test using a 2×2 contingency table was significantly higher among the COQ2 mutation carriers than among the noncarriers (p=0.018) (Table 8).

### 2-6. Intracellular CoQ10 Concentration in Lymphoblastoid Cells

The intracellular CoQ10 concentration in the lymphoblastoid cells obtained from MSA patients carrying V343A, lymphoblastoid cells obtained from MSA patients without variants, and lymphoblastoid cells obtained from controls without variants. The participants were classified into (1) MSA patients carrying two variant alleles (R337Q/V343A, R337X/V343A, and V343A/V343A), (2) 16 MSA patients carrying heterozygous V343A, (3) 133 MSA patients having no variant, and (4) 76 controls having no variant (Table 9).

TABLE 9

| | | | | | | Control |
|---|---|---|---|---|---|---|
| | MSA patients | | | | | subjects |
| | Variants | | | | | |
| | R337Q/ | R337X/ | V343A/ | | | |
| | V343A | V343A | V343A | V343A/wt | wt/wt | wt/wt |
| Number | 1 | 1 | 1 | 16 | 133 | 76 |
| Total CoQ₁₀/ | 2.19 | 2.58 | 1.86 | 3.38 | 3.41 | 3.48 |
| free | | | | (0.53) | (0.74) | (0.75) |
| cholesterol | | | | | | |
| Percent of | 62.9 | 74.1 | 53.4 | 97.1 | 98.0 | 100.0 |
| control mean | | | | | | |

Abbreviations:
MSA, multiple system atrophy;
CoQ₁₀, coenzyme q10.
Results on total CoQ₁₀/Tree cholesterol are presented as mean and standard deviation (nmol/mol) in parenthesis.

The intracellular CoQ10 concentration in the lymphoblastoid cells obtained from the MSA patients carrying two variant alleles was substantially lower than the concentration in the cells obtained from the controls having no variant. The intracellular CoQ10 concentration in the MSA patients having heterozygous V343A showed a decreasing tendency compared with that in the controls having no variant, but the difference was not significant. The intracellular CoQ10 concentration in the lymphoblastoid cells of the MSA patients having no COQ2 variant was equal to that of the controls having no COQ2 variant.

### 2-7. CoQ10 Concentration in Brain Tissue

Although the number of brain tissue samples available from MSA patients carrying CoQ2 variants was limited, the CoQ10 concentration in the frozen brain tissue from three patients having COQ2 variants (one patient with M78V-V343A/M78V-V343A and two patients with V343A/wt) and in the frozen brain tissue from a control having no variant was measured (FIG. 2C). The CoQ10 concentration in the MSA patients carrying homozygous M78V-V343A was significantly lower than that in the control having no variant.

### 3. Clinical Trial of Ubiquinol Intended for MSA Patients

### 3-1. Drug Used for Trial and Administration Method

A clinical trial of ubiquinol was carried out for one patient who had familial MSA and had a compound heterozygous R337X/V343A variant in the COQ2 gene (II-4 of FMSA_{—}12 shown in FIG. 1).

Stable powders containing 120 mg of ubiquinol (2-[(2E,6E,10E,14E,18E,22E,26E,30E,34E)-3,7,11,15,19,23,27,31,35,39-decamethyltetraconta-2,6,10,14,18,22,26,30,34,38-decaenyl]-5,6-dimethoxy-3-methylcyclohexa-2,5-diene-1,4-diol) provided by Kaneka Corporation were administered once in the morning from a gastrostoma tube at specified doses (600 mg, 840 mg, 1200 mg).

### 3-2. Design•Purpose of Trial

Non-blind exploratory clinical trial intended for the patient used as an example without providing a control was performed to investigate the following matters.

1. To confirm safety of high dose administration.
2. To study pharmacokinetics.
3. To determine dose of long-term administration from pharmacokinetics.
4. To investigate a clinical index.

### 3-3. Evaluation Items in Trial

The following are items evaluated in the clinical trial.

### (1) Primary Evaluation Item (Primary Endpoint)

Coenzyme Q10 concentration in the plasma, leucocyte, and spinal fluid

Presence or absence of adverse events

### (2) Secondary Evaluation Items (Secondary Endpoint)

UMSARS Part II (Evaluation of motor function)

Urinary 8-OHdG

Evaluation of oxygen metabolism by ¹⁵O-PET

### (3) Safety Evaluation Items

Liver function test (AST, ALT, γ-GTP, ALP, T.Bil)

The test is performed because it has been reported that administration of 300 mg/kg of coenzyme Q10 to rats slightly increased AST and ALT.

Subjective symptoms and objective findings by physical examination•neurological examination

Abnormalities in the results of various tests performed

### 3-4. Outline of Trial

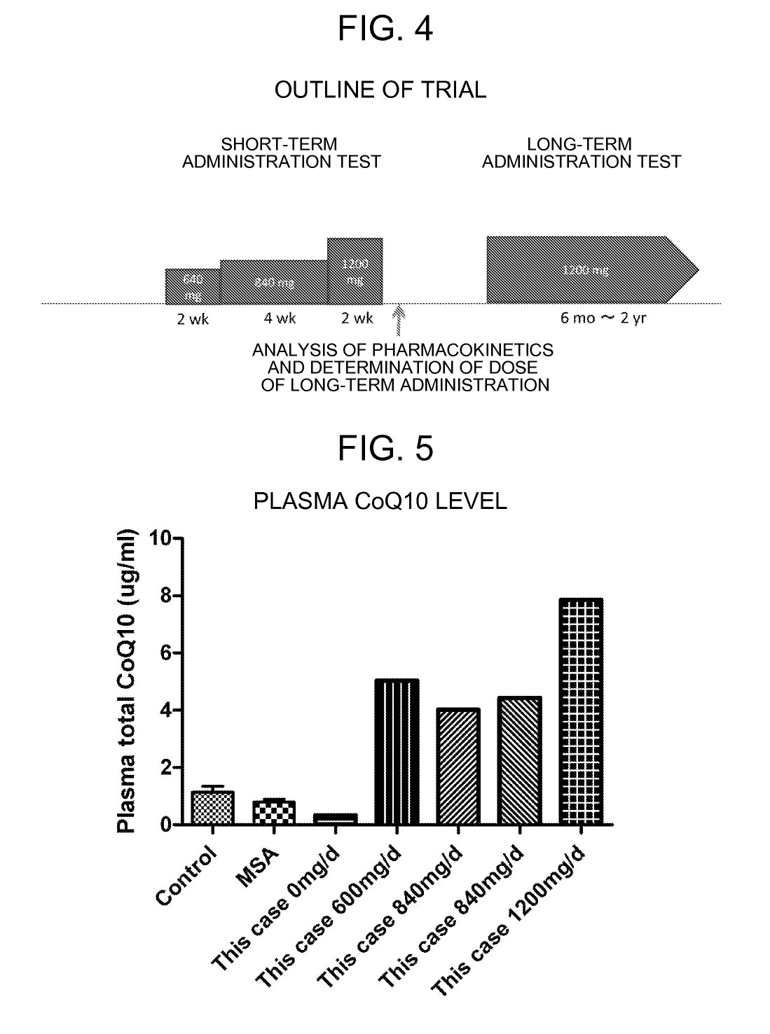

The outline of the trial is shown in FIG. 4.

### 3-5. Results of Short-Term Administration Trial

Adverse Events

During the trial term, presence or absence of subjective symptoms objective symptoms, and clinical trials (blood count and biochemical tests: AST, ALT, γ-GTP, ALP, T.Bil, BUN, Cre, Na, K, Cl, Glu, and CK) were observed with time.

Adverse events deemed attributable to the drug used for the trial did not occur.

### 3-6. Plasma CoQ10 Concentration

Measurement results of the blood CoQ10 concentration are shown in FIG. 5.

The plasma CoQ10 concentration of the base was low. When 1200 mg was administered, the concentration reached the plateau of the reported data. In the reported data on high dose administration of ubiquinone, the concentration was from 7.5 to 8.0 μg/ml and reached a plateau by the administration of 2400 mg or more of ubiquinone. In the present trial, the concentration reached 7.9 μg/ml by the administration of 1200 mg of ubiquinol, which was determined as a plateau based on the reported data.

### 3-7. The Amount of Total CoQ10 as Compared with Free Cholesterol in Mononuclear Cells

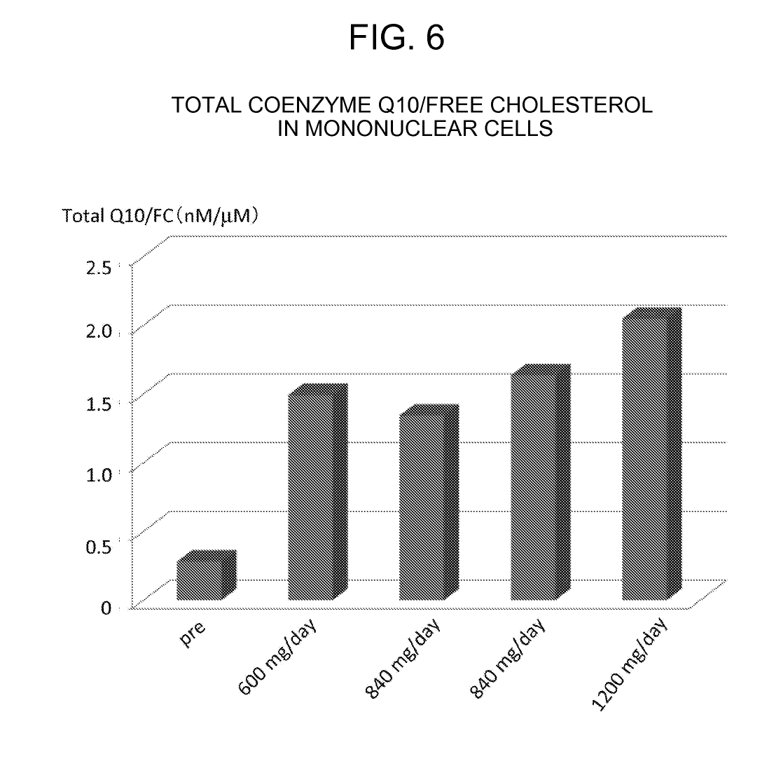

Measurement results of the total CoQ10/free cholesterol (nM/μM) in the mononuclear cells are shown in FIG. 6. It has been confirmed that administration of ubiquinol increased the total CoQ10/free cholesterol in the mononuclear cells dose-dependently.

### 3-8. CoQ10 Concentration in Spinal Fluid

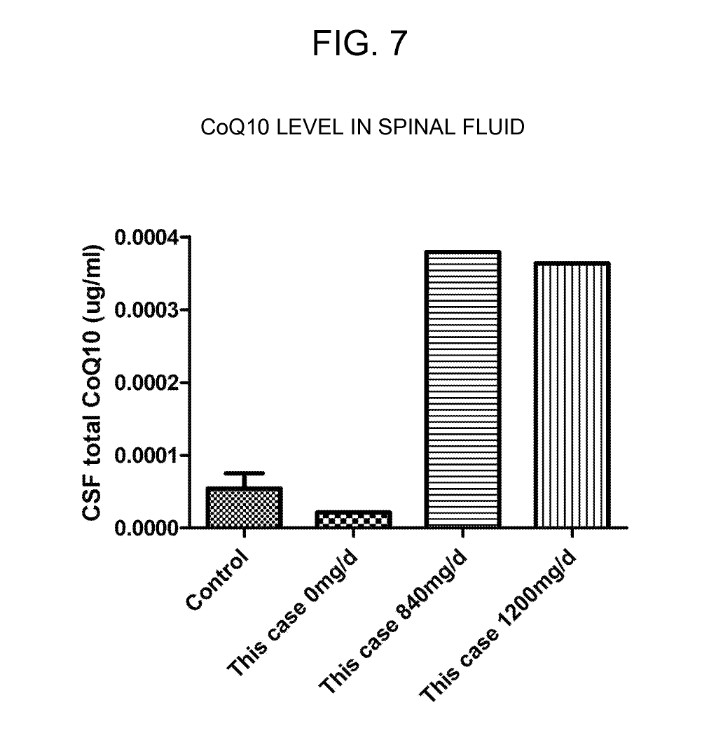

Measurement results of the CoQ10 concentration (μg/ml) in the spinal fluid are shown in FIG. 7. There is no report on a change of the CoQ10 concentration in the spinal fluid at the time of administration of ubiquinone•ubiquinol. The CoQ10 concentration in the spinal fluid was low in the base, but the concentration seemed to reach a plateau in the 840-mg or 1200-mg administered group.

### 3-9. Urinary 8-OHdG

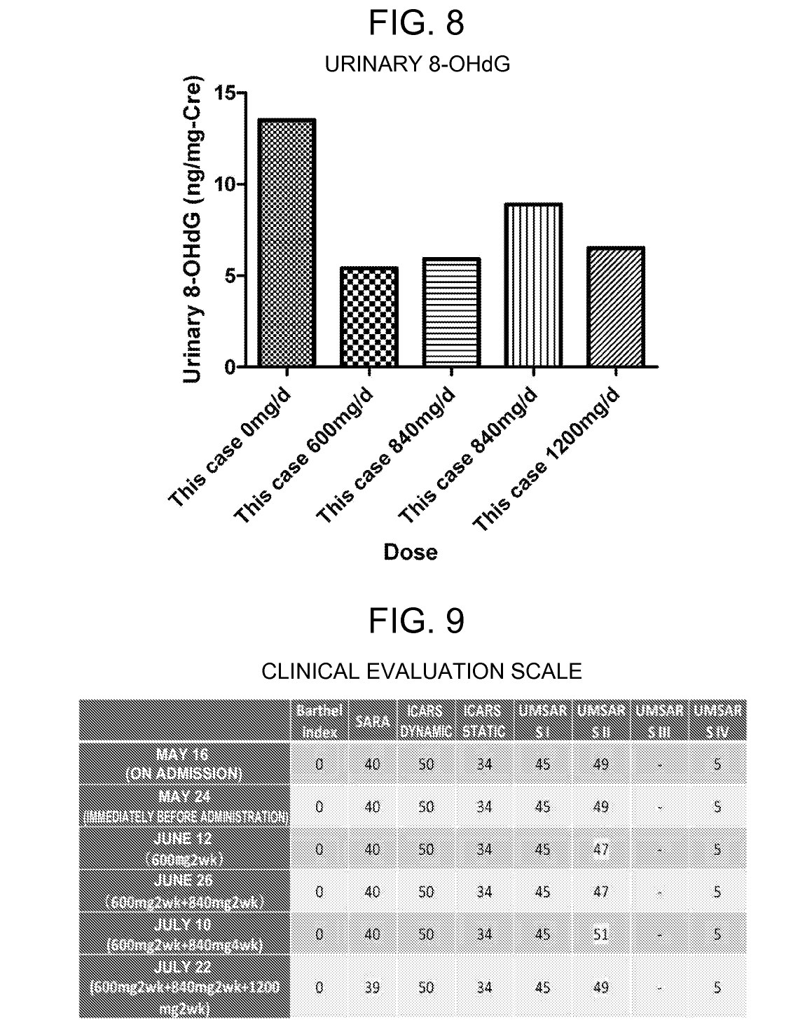

Measurement results of urinary 8-OHdG (ng/mg-Cre) are shown in FIG. 8. The urinary 8-OHdG level was high (mean: 8.4, n=500) in the base but it decreased by ubiquinol administration. The dose dependency was not clearly observed.

### 3-10. Clinical Evaluation Scale

Clinical evaluation scale is shown in FIG. 9. Changes deemed statistically significant were not found, though there were slight fluctuations. Impressions obtained were improvement in the response to calling from the doctor in charge or family (wife), improvement in the lifting of the upper limb, reduction in tremors of the extremities. It was however difficult to make a correct judgment because of complex factors such as subjective advice and rehabilitation effect in the hospital.

### 3-11. Cerebral Blood Flow Rate Enzyme Metabolic Rate

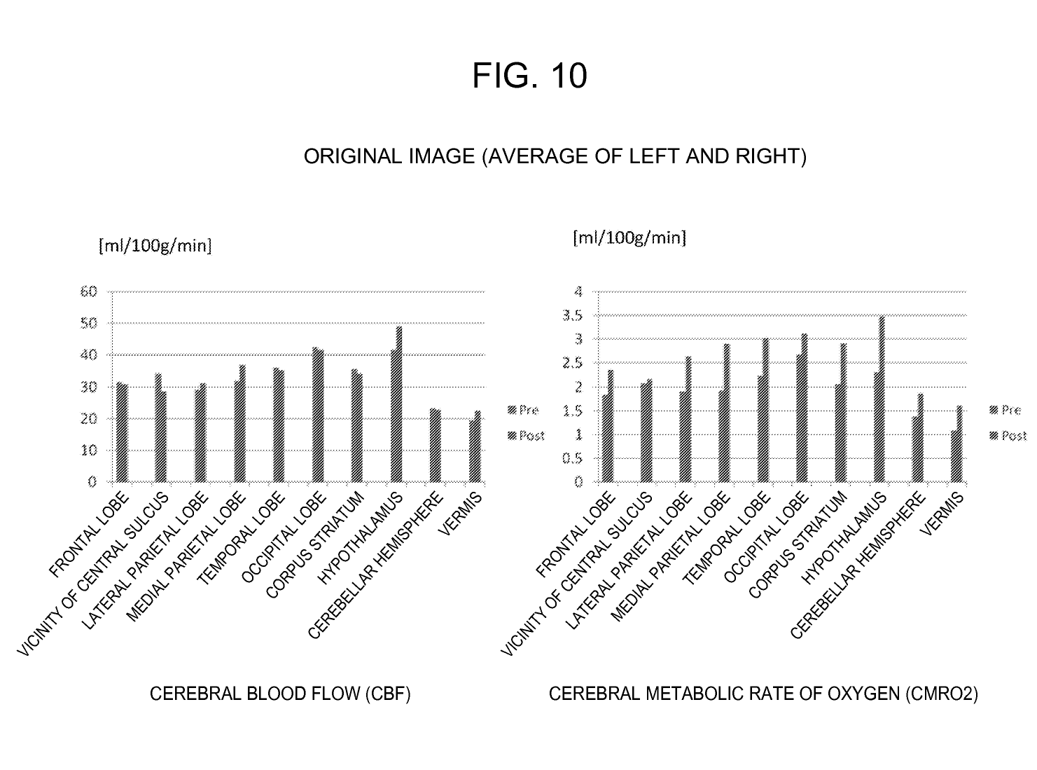

Measurement results of the cerebral blood flow rate and metabolic rate of oxygen are shown in FIG. 10. They each showed an increasing tendency after administration.

### 3-12. Conclusions

The present trial revealed the following points for the first time.

1. Even at high dose administration, ubiquinol has bioavailability higher than that of ubiquinone.
2. By the administration of 1200 mg of ubiquinol, the plasma CoQ10 reaches a plateau. The results agreeing with the plateau of the plasma CoQ10 observed in the prior research has been observed. This implies the transfer of the administered CoQ10 to the plasma.
3. It has been confirmed that administration of ubiquinol increases the CoQ10 content in the mononuclear cells of the peripheral blood. This shows the transfer of the administered CoQ10 into the cells.
4. It has been confirmed that administration of ubiquinol increases the CoQ10 concentration in the spinal fluid to a concentration exceeding the CoQ10 concentration in the spinal fluid of normal controls. This implies the transfer of the administered CoQ10 to the spinal fluid.
5. The findings in from 2 to 4 show that the administered CoQ10 can compensate reduction in the CoQ10.
5. During two-week administration of 1200 mg of ubiquinol, no adverse event is observed.
6. Evaluation of [¹⁵O]O₂ PET shows that a cerebral metabolic rate of oxygen was obviously improved by the administration. The possibility that replenishment with CoQ10 becomes a surrogate marker for evaluating the functional improvement of the central nervous system is suggested.

1. A method for testing the risk of multiple system atrophy of a test subject, comprising:
a step of detecting a variant that deteriorates biosynthesis of coenzyme Q10 in a sample collected from the test subject.

2. The method according to claim 1, wherein the variant that deteriorates biosynthesis of coenzyme Q10 is a variant that suppresses expression or function of para-hydroxybenzoate-polyprenyltransferase (coenzyme Q2).

3. The method according to claim 2, wherein the variant that suppresses expression or function of coenzyme Q2 is selected from a group consisting of P49H, S57T, R69H, M78V, I97T, P107S, S113F, T267A, S297C, R337Q, R337X, and V343A in SEQ ID NO: 1.

4. The method according to claim 2, wherein the variant that suppresses expression or function of coenzyme Q2 is V343A;

5. A test kit of multiple system atrophy, comprising at least one of the followings (i) to (iii):
(i) a nucleic acid that hybridizes with a region, in a coenzyme Q2 gene, containing a nucleic acid encoding an amino acid at a site selected from the group consisting of position 49, position 57, position 69, position 78, position 97, position 107, position 113, position 267, position 297, position 337, and position 343 of a coenzyme Q2 protein (SEQ ID NO: 1);
(ii) a primer set capable of amplifying a region, in the coenzyme Q2 gene, containing a nucleic acid encoding an amino acid at a site selected from the group consisting of position 49, position 57, position 69, position 78, position 97, position 107, position 113, position 267, position 297, position 337, and position 343 of the coenzyme Q2 protein (SEQ ID NO: 1); and
(iii) an antibody that binds, without cross-reactivity, only to either one of a coenzyme Q2 protein having at least one variant selected from the group consisting of P49H, S57T, R69H, M78V, I97T, P107S, S113F, T267A, S297C, R337Q, R337X, and V343A in SEQ ID NO: 1 or a wild type coenzyme Q2 protein.

6. A drug for the prevention or treatment of multiple system atrophy comprising coenzyme Q10;

7. A method of preventing or treating multiple system atrophy, comprising:
a step of administering coenzyme Q10;

8. A method of screening a drug for the prevention or treatment of multiple system atrophy, comprising:
a step of contacting candidate compounds with a cell and then incubating, and
a step of selecting a candidate compound that increases the amount of coenzyme Q10 in the cell.

9. A nucleic acid encoding coenzyme Q2 protein, comprising a V343A variant.


SEQUENCE LISTING (47 sequences)

SEQ ID NO: 1 - protein, 371 aa, Homo sapiens
MLGSRAAGFARGLRALALAWLPGWRGRSFALARAAGAPHGGDLQPPACPEPRGRQLSLSAAAVVDSAPRPLQPYLRLMRLDKPIGTWLLYLPCTWSIGLAAEPGCFPDWYMLSLFGTGAILMRGAGCTINDMWDQDYDKKVTRTANRPIAAGDISTFQSFVFLGGQLTLALGVLLCLNYYSIALGAGSLLLVITYPLMKRISYWPQLALGLTFNWGALLGWSAIKGSCDPSVCLPLYFSGVMWTLIYDTIYAHQDKRDDVLIGLKSTALRFGENTKPWLSGFSVAMLGALSLVGVNSGQTAPYYAALGAVGAHLTHQIYTLDIHRPEDCWNKFISNRTLGLIVFLGIVLGNLWKEKKTDKTKKGIENKIEN

SEQ ID NO: 2 - DNA, 19 bp, Artificial
  Synthesized primer.
tgaaggagggccacgagaa

SEQ ID NO: 3 - DNA, 22 bp, Artificial
  Synthesized primer.
cctagagtaagcgaccacgatg

SEQ ID NO: 4 - DNA, 20 bp, Artificial
  Synthesized primer.
ggggtcctttgtgatttgag

SEQ ID NO: 5 - DNA, 20 bp, Artificial
  Synthesized primer.
ttccatgctggatttctgtg

SEQ ID NO: 6 - DNA, 20 bp, Artificial
  Synthesized primer.
taccatgggccagtctcttc

SEQ ID NO: 7 - DNA, 25 bp, Artificial
  Synthesized primer.
tgtgtggtgagttacttacacttgc

SEQ ID NO: 8 - DNA, 25 bp, Artificial
  Synthesized primer.
ttgtcttaaagtatttcgtggtttc

SEQ ID NO: 9 - DNA, 25 bp, Artificial
  Synthesized primer.
atctctccataaaagtgtagtttgc

SEQ ID NO: 10 - DNA, 20 bp, Artificial
  Synthesized primer.
cactgaacacactccgatgc

SEQ ID NO: 11 - DNA, 22 bp, Artificial
  Synthesized primer.
tgctttctccttaatttggttc

SEQ ID NO: 12 - DNA, 22 bp, Artificial
  Synthesized primer.
tcaccgcttatggtatatctgc

SEQ ID NO: 13 - DNA, 20 bp, Artificial
  Synthesized primer.
tgccaggtaaacacagaggg

SEQ ID NO: 14 - DNA, 19 bp, Artificial
  Synthesized primer.
tttgctgttttctcctccg

SEQ ID NO: 15 - DNA, 27 bp, Artificial
  Synthesized primer.
aaatcttcatcttcaggttcttaattc

SEQ ID NO: 16 - DNA, 31 bp, Artificial
  Synthesized primer.
tcgcgcggggcctgcgggctgtggcactggc

SEQ ID NO: 17 - DNA, 29 bp, Artificial
  Synthesized primer.
agcccgcaggccccgcgcgaaccccgcgg

SEQ ID NO: 18 - DNA, 32 bp, Artificial
  Synthesized primer.
tgtggcactggcgtggctgctgggctggcggg

SEQ ID NO: 19 - DNA, 30 bp, Artificial
  Synthesized primer.
gcagccacgccagtgccacagcccgcaggc

SEQ ID NO: 20 - DNA, 32 bp, Artificial
  Synthesized primer.
cgggctggcggggccgctccctcgccctggcg

SEQ ID NO: 21 - DNA, 30 bp, Artificial
  Synthesized primer.
ggagcggccccgccagcccggcagccacgc

SEQ ID NO: 22 - DNA, 30 bp, Artificial
  Synthesized primer.
ttgcagccccccgcctgtcacgagccgcgc

SEQ ID NO: 23 - DNA, 29 bp, Artificial
  Synthesized primer.
gacaggcggggggctgcaagtcaccacgt

SEQ ID NO: 24 - DNA, 32 bp, Artificial
  Synthesized primer.
gccgcgcgggcgccagctcactttgtccgcgg

SEQ ID NO: 25 - DNA, 30 bp, Artificial
  Synthesized primer.
tgagctggcgcccgcgcggctcgggacagg

SEQ ID NO: 26 - DNA, 31 bp, Artificial
  Synthesized primer.
ggtggtggactctgcgccccaccccctgcag

SEQ ID NO: 27 - DNA, 30 bp, Artificial
  Synthesized primer.
ggggcgcagagtccaccaccgccgccgcgg

SEQ ID NO: 28 - DNA, 32 bp, Artificial
  Synthesized primer.
tgcagccgtacttgcgcctcgtgcggttggac

SEQ ID NO: 29 - DNA, 30 bp, Artificial
  Synthesized primer.
gaggcgcaagtacggctgcagggggcgggg

SEQ ID NO: 30 - DNA, 32 bp, Artificial
  Synthesized primer.
tttaccatgtacctggagcactggtttggcag

SEQ ID NO: 31 - DNA, 30 bp, Artificial
  Synthesized primer.
tgctccaggtacatggtaaatacagaagcc

SEQ ID NO: 32 - DNA, 32 bp, Artificial
  Synthesized primer.
cagctgaaccaggttgtttttcagattggtac

SEQ ID NO: 33 - DNA, 29 bp, Artificial
  Synthesized primer.
aaaacaacctggttcagctgccaaaccat

SEQ ID NO: 34 - DNA, 32 bp, Artificial
  Synthesized primer.
tccagattggtacatgctcttcctctttggca

SEQ ID NO: 35 - DNA, 29 bp, Artificial
  Synthesized primer.
agagcatgtaccaatctggaaaacaacct

SEQ ID NO: 36 - DNA, 32 bp, Artificial
  Synthesized primer.
ttttgattggtcttaagtcagcggctctgcgg

SEQ ID NO: 37 - DNA, 30 bp, Artificial
  Synthesized primer.
tgacttaagaccaatcaaaacatcatctct

SEQ ID NO: 38 - DNA, 32 bp, Artificial
  Synthesized primer.
tgagcctagtgggtgtgaactgtggacagact

SEQ ID NO: 39 - DNA, 30 bp, Artificial
  Synthesized primer.
gttcacacccactaggctcagtgcccccag

SEQ ID NO: 40 - DNA, 32 bp, Artificial
  Synthesized primer.
gttggaataaatttatctcccaccgaacactg

SEQ ID NO: 41 - DNA, 30 bp, Artificial
  Synthesized primer.
ggagataaatttattccaacaatcctcagg

SEQ ID NO: 42 - DNA, 32 bp, Artificial
  Synthesized primer.
gaataaatttatctccaaccaaacactgggac

SEQ ID NO: 43 - DNA, 30 bp, Artificial
  Synthesized primer.
ggttggagataaatttattccaacaatcct

SEQ ID NO: 44 - DNA, 32 bp, Artificial
  Synthesized primer.
ggaataaatttatctccaactgaacactggga

SEQ ID NO: 45 - DNA, 30 bp, Artificial
  Synthesized primer.
gttggagataaatttattccaacaatcctc

SEQ ID NO: 46 - DNA, 31 bp, Artificial
  Synthesized primer.
ccgaacactgggactaatagcttttttaggg

SEQ ID NO: 47 - DNA, 30 bp, Artificial
  Synthesized primer.
ctattagtcccagtgttcggttggagataa